SEQUENCE LISTING (4 sequences)

SEQ ID NO: 1 - DNA, 17 bp, Artificial
  primer
caggaaacagctatgac

SEQ ID NO: 2 - DNA, 22 bp, Artificial
  primer
cccctgtcatagctgtttcctg

SEQ ID NO: 3 - DNA, 24 bp, Artificial
  primer
tttttttgtcatagctgtttcctg

SEQ ID NO: 4 - DNA, 28 bp, Artificial
  primer
caggtttttttgtcatagctgtttcctg

(19) United States
(12) Patent Application Publication
(10) Pub. No.: US 2012/0108533 A1
(43) Pub. Date: May 3, 2012

(54) NOVEL PHOSPHATE MODIFIED NUCLEOSIDES USEFUL AS SUBSTRATES FOR POLYMERASES AND AS ANTIVIRAL AGENTS

(75) Inventors: Piet Herdewijn, Wezemaal (BE); Philippe Marlière, Mouscron (BE)

(73) Assignee: Katholieke Universiteit Leuven, K.U.Leuven R&D, Leuven (BE)

(21) Appl. No.: 13/266,954

(22) PCT Filed: Apr. 30, 2010
(86) PCT No.: PCT/EP2010/055942

§ 371 Date: Jan. 12, 2012

Related U.S. Application Data
(63) Continuation-in-part of application No. 12/549,117, filed on Aug. 27, 2009.
Application No. 12/549,117 is a continuation-in-part of application No. PCT/EP2008/001764, filed on Feb. 27, 2008.

(30) Foreign Application Priority Data
Feb. 27, 2007 (GB) 0703715.3
Feb. 27, 2007 (GB) 0703722.9
Sep. 17, 2007 (GB) 0718228.0
Sep. 17, 2007 (GB) 0718229.8
Apr. 30, 2009 (GB) 0907436.0
Dec. 11, 2009 (GB) 0921664.9

(51) Int. Cl. A61K 31/7064; C07H 19/20; C07H 21/02; A61P 31/18; C12P 19/34; C12P 21/06; C12P 19/28; A61P 31/12; C07H 19/048; C07H 21/04

(52) U.S. Cl. 514/49; 536/26.8; 536/26.7; 536/23.1; 435/91.1; 435/68.1; 435/85

(57) ABSTRACT

This invention provides phosphate-modified nucleosides represented by the structural formula: wherein W is O or S, and wherein B, R¹; R³ and R². are s defined herein. These compounds are useful as substrates for DNA/RNA polymerases, and as anti-viral agents in particular against HIV-1.

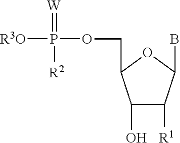

### FIELD OF THE INVENTION

The present invention relates to novel phosphate-modified nucleosides, such as carboxylyc acid containing phosphoramidate nucleosides. The present invention also relates to the phosphate-modified nucleosides as substrates for wild type and/or mutated DNA or RNA polymerases.

The present invention provides for the use of these novel phosphate-modified nucleosides for the production of oligonucleotides such as DNA or RNA and of polypeptides or proteins. The invention also relates to the use of these phosphate-modified nucleosides for growing or selecting specific micro-organisms, such as bacteria. The invention further provides for the use of these novel phosphate-modified nucleosides to treat or prevent viral infections and their use to manufacture a medicine to treat or prevent viral infections, particularly infections with viruses belonging to the HIV family.

The present invention furthermore relates to a method for the production of oligonucleotides, peptides or proteins by using said phosphate-modified nucleosides.

### BACKGROUND OF THE INVENTION

There has been significant progress in the design and synthesis of numerous nucleotide analogues bearing a modified nucleobase moiety or unnatural sugar and that are substrates for polymerases. Modifications at the phosphate moiety are introduced to increase the stability of a nucleotide toward enzymatic degradation or to mask the phosphate negative charge and facilitate its penetration into a cell. Common strategy in nucleotide prodrug design is protecting a phosphate moiety with a labile masking group. Removal of a masking group liberates a nucleoside monophosphate entity to be transformed into a nucleoside triphosphate (hereinafter referred as NTP), a substrate for intracellular enzymes. However, even after removal of the masking group, phosphorylation and activation of nucleoside monophosphate remains a problem due to substrate specificity of cellular kinases. Therefore, design of a nucleotide analogue that would allow bypassing the kinase activation pathway while behaving as a direct polymerase substrate would be a considerable challenge.

Treatment of certain viral infections has always been a challenging task due to ability of some viruses to integrate into a host's genome. Therefore, the viral enzymes that are critical for viral genome replication and integration are regarded as the most effective targets for the design of anti-viral agents.

A lot of attention has been given to studying mechanisms of action of Human Immunodeficiency Virus (type 1) (HIV-1) and developing specific inhibitors towards this very challenging and important target. One of the enzymes that are essential for the HIV replication is HIV reverse transcriptase (HIV RT). The function of this enzyme is to use a viral RNA genome and a reverse transcriptase to synthesize a double stranded DNA for integration into a host genome. Because this step is critical for the propagation of the viral infection, HIV reverse transcriptase (RT) is an excellent target for anti-viral treatment. Currently, two major classes of RT inhibitors (RTIs) exist and are administered for treatment of HIV infection. Non-nucleoside reverse transcriptase inhibitors (NNRTs) are a group of compounds that act through the allosteric inhibition by binding to a hydrophobic site, or a pocket in close proximity to the active site of HIV RT. The other group of RTIs is represented by nucleoside reverse transcriptase inhibitors (NRTIs) that bind directly to the active site and interfere with the polymerization reaction and DNA synthesis.

Nucleoside reverse transcriptase inhibitors are designed to be recognized as substrates for RT and incorporated into a growing strand for further termination of chain elongation. Inhibition of reverse transcriptase activity and chain termination by NRTIs is achieved by introduction of structural modifications to the sugar moiety. The elongation of the DNA strand by a polymerase requires a nucleophilic attack of the 3′-OH group to the α phosphorus atom of an incoming nucleotide. Therefore, nucleoside analogs that lack the 3′-OH group or have it substituted with other functional groups (for instance, N₃, F, H) not capable of the nucleophilic attack and formation of phosphodiester bond would act as chain terminators.

Termination of DNA or RNA synthesis with nucleoside analogues is a common and one of the most efficient strategies in the treatment of viral infections, regardless of various side effects and cell toxicity. The therapeutically active form of a nucleoside analogue is a nucleoside triphosphate. However, at the physiological pH nucleoside triphosphates are negatively charged molecules and thus they can not penetrate cellular membranes. Hence, RT inhibitors are usually administered as biologically inactive free nucleosides or as monophosphate pro-drugs where a phosphate group is masked with a lipophilic group.

There are three steps of kinase-mediated activation of anti-viral nucleosides. At first, transformation to a monophosphate derivative takes place through the action of a cytoplasmic nucleoside kinase (for instance, thymidine kinase and deoxycytidine kinase). Furthermore, a nucleoside 5′-monophosphate kinase catalyzes the conversion of a nucleoside monophosphate to a nucleoside diphosphate. Finally, a diphosphate derivative is phosphorylated by a nucleoside 5′-diphosphate kinase (NDK) to provide an anti-viral nucleoside analog in its activated (phosphorylated) form. The efficiency of phosphorylation depends on substrate specificity of kinases. For instance, in the case of the AZT phosphorylation cascade, conversion from the nucleoside monophosphate to the nucleoside diphosphate becomes a rate limiting step as thymidylate kinase (TMPK) catalyzes this conversion significantly slower than in the case of the natural substrate (TMP). The consequences of this inefficiency are accumulation of AZTMP in the cytosol and decreased therapeutic concentration of AZTTP, the activated nucleoside form. However, it was determined that high levels of AZTMP have an inhibitory effect on thymidylate kinase by competing with its natural substrate (TMP) and resulting in reduced levels of TDP and TTP. Moreover, increased levels of AZT and its phosphorylated derivatives also affect other enzymes of the de novo dNTPs synthesis resulting in skewed natural nucleotide concentrations.

Therefore, administration of free NRTIs, which often relies on intracellular phosphorylation and activation, has significant drawbacks. One of the possible solutions is a prodrug or pronucleotide approach. In the prodrug approach, the monophosphate moiety is “masked” with a labile functional group which also serves to facilitate passage of a “masked” nucleotide inside the cell. Once inside the cell, a masking group is removed either enzymatically or through chemical activation. Removal of the masking group affords a free nucleoside monophosphate intracellularly where it can be further phosphorylated by TMPK and NDK. Thus, although the prodrug approach facilitates delivery of an inhibitory nucleoside inside the cell and eliminates the need for initial phosphorylation by a nucleoside kinase, phosphorylation by TMPK and NDK are still required.

Besides delivery and bio-distribution challenges, another drawback that is often associated with anti-viral therapy is emergence of resistant strains. In the case of HIV-1, the drug resistance is developed by appearance of mutations that would allow HIV RT to discriminate NRTIs for natural nucleotides or remove an incorporated unnatural nucleobase by excision reactions. It has also been shown for herpes simplex virus (HSV) that reduction in anti-herpetic activity of acyclovir, a drug activated by thymidine kinase phosphorylation and commonly used for treatment of HSV infections, is mostly associated with thymidine kinase dependent resistance. Established strategies to manage acyclovir-resistant HSV infections include administration of anti-viral drugs acting directly on a viral DNA polymerase (foscarnet, cidifovir) or by modulating immune response of a patient. However, the later approach is not always feasible and the former one could worsen patient's condition since these medications impose a significant level of toxicity. WO 00/47591 discloses phosphoramidates of 4-(6-amino-purin-9-yl)-2-cyclopentene-1-methanol being useful in treating viral infections. These compounds however do not include a ribose or deoxyribose sugar moiety as required in any nucleoside.

WO 2007/020193 discloses antiviral nucleoside phosphoramidates wherein one carbon atom of the ribose or deoxyribose sugar moiety is substituted with a group selected from the group consisting of azido, ethynyl and chloroethenyl, and wherein the nitrogen atom of said phosphoramidate is substituted with hydrogen or C₁₋₃ alkyl. These compounds however do not include any pending aryl or carboxylic acid groups on the phosphoramidate moiety attached to the nucleoside or deoxynucleoside.

J. Org. Chem. (2005) 70:1100-1103 discloses (scheme 2) the use of resin-bound phosphitylating reagents to yield monophosphorylated unprotected nucleosides such as thymine, uridine and adenosine. The document however does not teach nucleoside or deoxynucleoside phosphoramidates or phosphates including any pending aryl or carboxylic acid groups.

J. Med. Chem. (1992) 35:2728-2735 discloses (compounds 12 and 13) the 5′-phenyl phosphates of 2′,3′-didehydro-2′,3′-dideoxyadenosine and 2′,3′-didehydro-2′,3′-dideoxycytidine which in serum-containing medium can function as pro-drugs of the antiviral nucleosides which are released before their introduction into cells. The document however does not teach any nucleoside or deoxynucleoside phosphoramidates or any nucleoside or deoxynucleoside phosphates including pending aryl groups linked to the phosphate group via a short alkylene chain, or pending carboxylic acid groups.

Antiviral Chemistry and Chemotherapy (1998) 9; 1-8 discloses (compound 11) 5′-[(hydroxyl)(phenyl)phosphinyl]thymidine which was tested for herpes simplex virus inhibition. The document however does not teach nucleoside or deoxynucleoside phosphoramidates or phosphates including any pending aryl or carboxylic acid groups.

French Patent No. 2,781,229 discloses on the one hand 2′,3′-didehydro-2′,3′-dideoxythymidine-5′-dibenzylphosphate (example 1) and 2′,3′-didehydro-2′,3′-dideoxythymidine-5′-diethylphosphate (example 3) made from dibenzylphosphite or diethylphosphite respectively, and on the other hand a family of 5′-H-phosphonates of 2′,3′-didehydro-2′,3′-dideoxynucleosides made from O-benzyl H-phosphonate such as 2′,3′-didehydro-2′,3′-dideoxythymidine-5′-benzyl H-phosphonate (example 4). The document however does not teach any nucleoside or deoxynucleoside phosphoramidates or any nucleoside or deoxynucleoside phosphates including pending carboxylic acid groups.

Antiviral Chemistry and Chemotherapy (2002) 13; 101-114 discloses 2′,3′-didehydro-2′,3′-dideoxythymidine-5′-dibenzylphosphate and 2′,3′-didehydro-2′,3′-dideoxythymidine-5′-di(α-methylbenzylphosphate (compounds 4 and 5) being made from bis(benzyl)-N,N-diisopropylphosphoramidite. The document however does not teach any nucleoside or deoxynucleoside phosphoramidates or any nucleoside or deoxynucleoside phosphates including pending carboxylic acid groups.

JP 2004-043371 discloses the 5′-dibenzylphosphate of a ribofuranosyl-pyrazine carboxamide. The document however does not teach any nucleoside or deoxynucleoside phosphoramidates or any nucleoside or deoxynucleoside phosphates including pending carboxylic acid groups.

Bioorg. Med. Chem. (2006) 14:1924-1934 discloses 5′-aryl H-phosphonates and 5′-aryl α-hydroxy(aryl)methanephosphonates of 2′,3′-dideoxyinosine or 3′-azido-2′,3′-dideoxythymidine wherein aryl may be phenyl, 4-methylphenyl, 4-methoxyphenyl, 2,6-dimethylphenyl, 4-chlorophenyl, 4-nitrophenyl or pyridin-3-yl. The document however does not teach any nucleoside or deoxynucleoside phosphoramidates or any nucleoside or deoxynucleoside phosphates including pending aryl or carboxylic acid groups.

WO 2008/104408 discloses nucleoside phosphates, phosphorothioates and phosphoramidates including a pending imidazolyl or carboxylic group attached to their O or S atom or NH group.

WO 01/34622 discloses (page 8 and claim 13) nucleoside derivatives being both:

substituted at carbon 2 with a R³ or OR³ group, and

phosphorylated with a group PXYR,

wherein each of X and Y may be O, S, OR or NR¹R²
wherein R is a hydrophobic group having 1 to 18 carbon atoms, and
wherein each of R¹, R² and R³ is as defined by R.
However no individual compound disclosed at pages 13-16 falls under this definition. Scheme 1 and tables 1-2 disclose deoxynucleoside derivatives being phosphorylated with a group P(S)(OH)OR (i.e. phosphorothioates) wherein R is phenylmethyl, phenylethyl, naphthylmethyl, alkyl, cycloalkyl, cycloalkyl-alkyl, cycloalkenyl-alkyl or heterocyclyl. This document however does not teach any nucleoside or deoxynucleoside phosphoramidates or any nucleoside or deoxynucleoside phosphates including pending aryl or carboxylic acid groups.

Bioorganichevskaya Khimiya (1987) 13:565-7 discloses guanosine 5′-[hydrogen[[4-[(2-chloroethyl)methylamino]phenyl]methyl]phosphoramidate. This document however does not teach any nucleoside or deoxynucleoside phosphates including pending aryl or carboxylic acid groups, or any nucleoside or deoxynucleoside phosphoramidates including pending carboxylic acid groups or pending aryl groups directly attached to the nitrogen atom of said phosphoramidate.

Nucleic Acids Research (1989) 17:8979-89 discloses N,N-dimethylguanosine 5′-(S-phenyl hydrogen phosphorothioate. This document however does not teach any nucleoside or deoxynucleoside phosphoramidates, or any nucleoside or deoxynucleoside phosphates including pending carboxylic acid groups or pending aryl groups linked to the phosphate group via a short alkylene chain.

Nucleosides & Nucleotides (1987) 6:913-34 discloses adenosine 5′-(hydrogen phenylphosphoramidate) and adenosine 5′-[hydrogen (phenylmethyl)-phosphoramidate]. This document however does not teach any nucleoside or deoxynucleoside phosphates including pending carboxylic acid groups or aryl groups, or any nucleoside or deoxynucleoside phosphoramidates including pending carboxylic acid groups.

Nucleic Acids Research (1974) discloses uridine 5′-[hydrogen (4-chlorophenyl)phosphoramidate], uridine 5′-[hydrogen (4-bromophenyl)-phosphoramidate] and uridine 5′-[hydrogen (4-iodophenyl)phosphoramidate]. This document however does not teach any nucleoside or deoxynucleoside phosphates including pending carboxylic acid groups or aryl groups, or any nucleoside or deoxynucleoside phosphoramidates including pending carboxylic acid groups.

Therefore, considering all aforementioned aspects of therapy directed to inhibit viral polymerases and reverse transcriptases, a nucleotide analogue that would not depend on activation by nucleoside/nucleotide kinases whilst serving as a natural substrate mimic, would be of a great interest. Also, there is still a need for the development of novel phosphate-modified nucleosides that meet the requirements for successful polymerase recognition, including good chelating properties and spatial features to form stable enzyme-substrate complexes, and whereby the incorporation reaction is not stalled.

### SUMMARY OF THE INVENTION

The present invention provides novel phosphate-modified nucleosides which can act as substrates of DNA- or RNA-polymerases and/or as antiviral agents.

The present invention provides novel phosphate-modified nucleosides that can be used as alternative (compared to natural NTPs or dNTPs) efficient substrates for DNA- or RNA-polymerases. In a particular embodiment, these phosphate-modified nucleosides are such that the pyrophosphate group of nucleosides/nucleotides is replaced by an easily leaving group, more particularly a leaving group in a nucleotidyl transfer mechanism. Preferably the leaving group comprises at least a pending aryl (e.g. phenyl) group or, when the phosphate modification results into a phosphoramidate, at least two carboxylic acids attached (more preferably separately attached) to the nitrogen atom thereof. In a specific embodiment of the invention, this leaving group includes or is a carboxylic acid-containing group coupled to the nucleoside by a phosphoramide binding moiety, yet more particularly this carboxylic acid containing group comprises at least two carboxylic acid groups being each linked via 0, 1, 2 or 3 CH₂-groups to the N of the phosphoramide binding moiety. In another specific embodiment of the invention, this leaving group is a aryl-containing group coupled to the nucleoside by a phosphoramide, phosphate or phosphorothioate binding moiety, yet more particularly this aryl-containing group comprises one, two or three phenyl groups being each linked via 0, 1, 2 or 3 methylene (CH₂) groups to said phosphoramide, phosphate or phosphorothioate binding moiety. Preferentially said phenyl group(s) is (are) substituted with one or two carboxylic acid groups.

### DETAILED DESCRIPTION OF THE INVENTION

According to a first broad aspect, the present invention encompasses modified nucleosides represented by the structural formula (A):

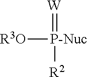

wherein
Nuc is a natural nucleoside or a nucleoside analogue, wherein said natural nucleoside or nucleoside analogue can be non-substituted or substituted as defined below;
R³ is selected from the group consisting of hydrogen, C₁₋₆ alkyl, C₃₋₆ cycloalkyl, aryl-C₁₋₆ alkyl, and 2-cyanoethyl; wherein any one of alkyl, cycloalkyl or arylalkyl may optionally be substituted with 1, 2 or 3 substituents independently selected from the group consisting of halogen, OH, C₁₋₆ alkoxy, trifluoromethyl, trifluoromethoxy, nitro, cyano and amino;
W is O or S;
R² is represented by the structural formula (V):

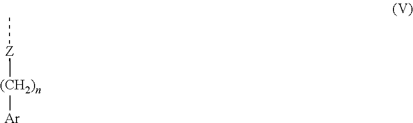

wherein
dotted lines represent the point of attachment of Z to the phosphorous atom P of the structural formula (A);
n is 0, 1 or 2;
Z is selected from the group consisting of O; S; NH and NCH₃; and
Ar is an aryl group as defined below,
   provided that when W is S and Z is O, n is not 1 or 2,
   and stereoisomers, pharmaceutically acceptable salts and pro-drugs thereof,
   or R² is represented by the structural formula (II)

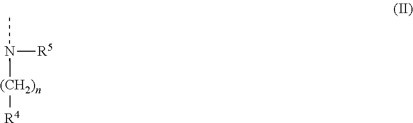

wherein
dotted lines represent the point of attachment of N to the phosphorous atom of the structural formula (A);
n is 0, 1, 2, or 3;
R⁴ is an aryl group or COOR⁶, wherein R⁶ is hydrogen or C₁₋₆ alkyl or benzyl; and
R⁶ is a group represented by the structural formula (III):

—(CH₂)ₘ—R¹¹ (III)
wherein
   the dotted line represents the point of attachment to the nitrogen atom of the structural formula (II);
   m is 0, 1, 2, or 3; and
   R¹¹ is selected from the group consisting of aryl, imidazolyl
and COOR⁶ wherein R⁶ is hydrogen or C₁₋₆ alkyl or benzyl;
or R⁵ is a group represented by the structural formula (IV)

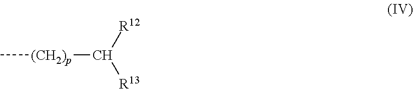

wherein
   the dotted line represents the point of attachment to the nitrogen atom of the structural formula (II);
   p is 0, 1, 2, or 3; and
   each of R¹² and R¹³ is independently selected from the group consisting of aryl; hydrogen; (CH₂)_{q}-imidazolyl; and (CH₂)_{q}—COOR⁶, wherein R⁶ is hydrogen or C₁₋₆ alkyl or benzyl, and wherein q is 0, 1 or 2, provided that if p is 0, R¹² and R¹³ are not both hydrogen;
      and stereoisomers, pharmaceutically acceptable salts and pro-drugs thereof, provided that said modified nucleoside is not:
2′,3′-didehydro-2′,3′-dideoxyadenosine-5′-phenyl phosphate,
2′,3′-didehydro-2′,3′-dideoxycytidine-5′-phenyl phosphate,
ribofuranosyl-pyrazine carboxamide-5′-dibenzylphosphate,
2′,3′-didehydro-2′,3′-dideoxythymidine-5′-dibenzylphosphate,
2′,3′-didehydro-2′,3′-dideoxythymidine-5′-di(α-methylbenzyl)phosphate,
guanosine 5′-[hydrogen[[4-[(2-chloroethyl)methylamino]phenyl]methyl]-phosphoramidate,
N,N-dimethylguanosine 5′-(S-phenyl hydrogen phosphorothioate),
adenosine 5′-(hydrogen phenylphosphoramidate),
adenosine 5′-[hydrogen (phenylmethyl)phosphoramidate,
uridine 5′-[hydrogen (4-chlorophenyl)phosphoramidate],
uridine 5′-[hydrogen (4-bromophenyl)phosphoramidate], or
uridine 5′-[hydrogen (4-iodophenyl)phosphoramidate.

According to a specific embodiment of this first aspect of the invention, said natural nucleoside or nucleoside analogue (Nuc) is coupled via its 5′ position (referring to the standard numbering of atoms for cyclic sugar moieties) to the phosphorus atom P in the structural formula (A).

According to another specific embodiment of this first aspect of the invention, the modified nucleoside is one wherein R² is represented by the structural formula (II), wherein R⁵ is represented by the structural formula (III), and wherein at least one of R⁴ and R¹¹ is COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein R⁴ and R¹¹ are both COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least one of R¹² and R¹³ is COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein R¹² and R¹³ are both COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least one of R⁴, R¹² and R¹³ is COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least two of R⁴, R¹² and R¹³ are COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein all of R⁴, R¹² and R¹³ are COOR⁶.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein at least one of R⁴ and R¹¹ is COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein R⁴ and R¹¹ are both COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least one of R¹² and R¹³ is COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein R¹² and R¹³ are both COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least one of R⁴, R¹² and R¹³ is COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein at least two of R⁴, R¹² and R¹³ are COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (IV), and wherein all of R⁴, R¹² and R¹³ are COOH.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein m=n.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein m=n=1.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein m=n=2.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein m differs from n.

According to another specific embodiment of this first aspect of the invention, the modified nucleoside is one wherein Nuc, W and R³ are as broadly defined hereinabove, wherein R² is represented by the structural formula (II), wherein R⁵ is represented by the structural formula (III), and wherein m=1 and n=2, or wherein m=2 and n=1.

According to another specific embodiment of this first aspect of the invention, the modified nucleoside is one wherein Nuc, W and R³ are as broadly defined hereinabove, wherein R² is represented by the structural formula (V), wherein n=0, and wherein Ar is a phenyl or substituted phenyl group.

According to another specific embodiment of this first aspect of the invention, the modified nucleoside is one wherein Nuc, W and R³ are as broadly defined hereinabove, wherein R² is represented by the structural formula (V), wherein n=1, and wherein Ar is a phenyl or substituted phenyl group.

According to another specific embodiment of this first aspect of the invention, the modified nucleoside is one wherein Nuc, W and R³ are as broadly defined hereinabove, wherein R² is represented by the structural formula (V), wherein n=2, and wherein Ar is a phenyl or substituted phenyl group.

According to another specific embodiment of this first aspect of the invention, the modified nucleoside is one wherein Nuc, W and R³ are as broadly defined hereinabove, wherein R² is represented by the structural formula (II), and wherein at each occurrence of R⁶ in the definitions of R⁴ and R⁵, R⁶ is hydrogen or C₁₋₆ alkyl.

A second more specific aspect of the present invention relates to modified nucleosides represented by the structural formula (I):

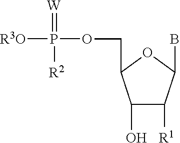

wherein
B is a pyrimidine or purine base, or an analogue thereof (such as defined below), optionally substituted with one or two substituents independently selected from the group consisting of halogen, hydroxyl, sulfhydryl, methyl, ethyl, isopropyl, amino, methylamino, ethylamino, trifluoromethyl and cyano;
R¹ is H or OH;
R³ is selected from the group consisting of hydrogen, C₁₋₆ alkyl, C₃₋₆ cycloalkyl, aryl-C₁₋₆ alkyl and 2-cyanoethyl, wherein said C₁₋₆ alkyl, C₃₋₆ cycloalkyl or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;
W is O or S; and
R² is represented by the structural formula (V):

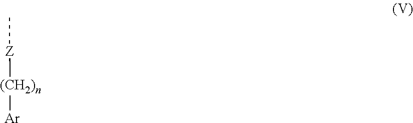

wherein
dotted lines represent the point of attachment of Z to the phosphorous atom P of the structural formula (I);
n is 0, 1 or 2;
Z is selected from the group consisting of O; S; NH and NCH₃; and
Ar is an aryl group as defined below,
   provided that when W is S and Z is O, n is not 1 or 2,
   and stereoisomers, pharmaceutically acceptable salts and pro-drugs thereof,
   or R² is represented by the structural formula (II):

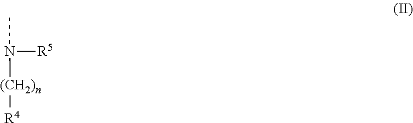

wherein
dotted lines represent the point of attachment of N to the phosphorous atom P of the structural formula (I);
n is 0, 1, 2, or 3;
R⁴ is selected from the group consisting of aryl, imidazolyl and COOR⁶ wherein R⁶ is H or C₁₋₆ alkyl or benzyl;
R⁵ is a group represented by the structural formula (III):

—(CH₂)ₘ—R¹¹ (III)

wherein
the dotted line represents the point of attachment to the nitrogen atom of the structural formula (II);
m is 0, 1, 2, or 3; and
R¹¹ is selected from the group consisting of: aryl, imidazolyl and COOR⁶ wherein R⁶ is hydrogen or C₁₋₆ alkyl or benzyl;
   or R⁵ is a group represented by the structural formula (IV):

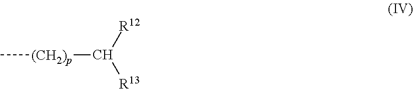

wherein
the dotted line represents the point of attachment to the nitrogen atom of the structural formula (II);
p is 0, 1, 2, or 3; and
each of R¹² and R¹³ is independently selected from the group consisting of aryl; hydrogen; (CH₂)_{q}-imidazolyl; and (CH₂)_{q}—COOR⁶, wherein R⁶ is hydrogen or C₁₋₆ alkyl or benzyl, and wherein q is 0, 1 or 2, provided that if p is 0, R¹² and R¹³ are not both hydrogen,
   and stereoisomers, pharmaceutically acceptable salts and pro-drugs thereof, provided that said modified nucleoside is not:
2′,3′-didehydro-2′,3′-dideoxyadenosine-5′-phenyl phosphate,
2′,3′-didehydro-2′,3′-dideoxycytidine-5′-phenyl phosphate,
ribofuranosyl-pyrazine carboxamide-5′-dibenzylphosphate,
2′,3′-didehydro-2′,3′-dideoxythymidine-5′-dibenzylphosphate,
2′,3′-didehydro-2′,3′-dideoxythymidine-5′-di(α-methylbenzyl)phosphate,
guanosine 5′-[hydrogen[[4-[(2-chloroethyl)methylamino]phenyl]methyl]-phosphoramidate,
N,N-dimethylguanosine 5′-(S-phenyl hydrogen phosphorothioate),
adenosine 5′-(hydrogen phenylphosphoramidate),
adenosine 5′-[hydrogen (phenylmethyl)phosphoramidate,
uridine 5′-[hydrogen (4-chlorophenyl)phosphoramidate],
uridine 5′-[hydrogen (4-bromophenyl)phosphoramidate], or
uridine 5′-[hydrogen (4-iodophenyl)phosphoramidate.

In each of the structural formulae (A) and (I), W is preferably O (oxygen) but it can be replaced by S by chemical reactions known in the art.

According to a particular embodiment of the present invention, i.e. with respect to the structural formula (A) or the structural formula (I), the molecular weight of the group R₂ is not above 500.

In a particular embodiment, the second aspect of the present invention relates to phosphate-modified nucleosides represented by the structural formula (I) wherein R¹, R², R³ and W have any of the values as described herein-above, and wherein B is adenine; guanine; cytosine; thymine; uracil, or a substituted uracil as described below.

In another particular embodiment, the second aspect of the present invention relates to phosphate-modified nucleosides represented by the structural formula (I) wherein R¹, R³ and W have any of the values as described herein-above, wherein R² is represented by the structural formula (V), and wherein B is a pyrimidine base substituted with one or two substituents independently selected from the group consisting of halogen, hydroxyl, sulfhydryl, methyl, ethyl, amino and methylamino.

In another particular embodiment, the second aspect of the present invention relates to phosphate-modified nucleosides represented by the structural formula (I) wherein R¹, R³ and W have any of the values as described herein-above, wherein R² is represented by the structural formula (V), and wherein B is a purine base substituted with one or two substituents independently selected from the group consisting of halogen, hydroxyl, sulfhydryl, methyl, ethyl, amino and methylamino.

In another particular embodiment, the second aspect of the present invention relates to phosphate-modified nucleosides represented by the structural formula (I) wherein R¹, R³ and W have any of the values as described herein-above, wherein R² is represented by the structural formula (II), wherein n, R⁴ and R⁵ have any of the values as described herein-above, and wherein B is a pyrimidine base substituted with one or two substituents independently selected from the group consisting of halogen, hydroxyl, sulfhydryl, methyl, ethyl, amino and methylamino.

In another particular embodiment, the second aspect of the present invention relates to phosphate-modified nucleosides represented by the structural formula (I) wherein R¹, R³ and W have any of the values as described herein-above, wherein R² is represented by the structural formula (II), wherein n, R⁴ and R⁵ have any of the values as described herein-above, and wherein B is a purine base substituted with one or two substituents independently selected from the group consisting of halogen, hydroxyl, sulfhydryl, methyl, ethyl, amino and methylamino.

In another particular embodiment, the second aspect of the present invention relates to the phosphate-modified nucleosides represented by the structural formula (I) wherein B, R¹, R² and W have any of the values as described herein, and wherein R³ is hydrogen.

In another particular embodiment, the second aspect of the present invention relates to the phosphate-modified nucleosides represented by the structural formula (I) wherein B, R¹, R² and R³ have any of the values as described herein, and wherein W is O.

In another particular embodiment, the second aspect of the present invention relates to the phosphate-modified nucleosides represented by the structural formula (I) wherein B, R², R³ and W have any of the values as described herein, and wherein R¹ is hydrogen.

In another particular embodiment, the second aspect of the present invention relates to phosphate-modified nucleoside represented by the structural formula (I) wherein B, R², R³ and W have any of the values as described herein, and wherein R¹ is OH.

In another particular embodiment, the se aspect of the present invention also relates to phosphate-modified nucleosides represented by the structural formula (I) wherein B, R¹, R³ and W have any of the values described herein-above, wherein R² is represented by the structural formula (V), and wherein n is 0.

In another particular embodiment, the se aspect of the present invention also relates to phosphate-modified nucleosides represented by the structural formula (I) wherein B, R¹, R³ and W have any of the values described herein-above, wherein R² is represented by the structural formula (V), and wherein n is 1.

In another particular embodiment, the se aspect of the present invention also relates to phosphate-modified nucleosides represented by the structural formula (I) wherein B, R¹, R³ and W have any of the values described herein-above, wherein R² is represented by the structural formula (II), and wherein n is 0.

In another particular embodiment, the se aspect of the present invention also relates to phosphate-modified nucleosides represented by the structural formula (I) wherein B, R¹, R³ and W have any of the values described herein-above, wherein R² is represented by the structural formula (II), and wherein n is 1.

In another particular embodiment, the second aspect of the present invention relates to phosphate-modified nucleosides represented by the structural formula (I) wherein B, R¹, R³ and W have any of the values described herein, wherein R² is represented by the structural formula (II), wherein R⁵ is represented by the structural formula (III), and wherein m is 1.

In another particular embodiment, the second aspect of the present invention relates to phosphate-modified nucleosides represented by the structural formula (I) wherein B, R¹, R³ and W have any of the values described herein, wherein R² is represented by the structural formula (II), wherein R² is represented by the structural formula (IV), and wherein p is 0.

In another particular embodiment, the second aspect of the present invention relates to phosphate-modified nucleosides represented by the structural formula (I) wherein R¹, R², R³ and W have any of the values described herein, and wherein B is a pyrimidine or purine base analogue as described in the Definitions section below, in particular 5-azapyrimidine, 5-azacytosine, 7-deazapurine, 7-deazaadenine, 7-deazaguanine or 7-deaza-8-azapurine.

In another particular embodiment of the foregoing, the second aspect of the present invention relates to phosphate-modified nucleosides represented by the structural formula (I) wherein B, R¹, R³ and W have any of the values as described herein, and wherein R² is a nitrogen-linked carboxylic acid-containing group coupled by a phosphoramide binding, and in a more particular embodiment of the foregoing said carboxylic acid containing group comprises two carboxylic acid groups linked via 0, 1, 2 or 3 CH₂-groups to the N of the phosphoramide binding, and in an even more particular embodiment of the foregoing R² is N(CH₂—COOH)₂.

In another particular embodiment of the foregoing, the first aspect of the present invention relates to phosphate-modified nucleosides represented by the structural formula (A) wherein Nuc, R¹, R³ and W have any of the meanings as described herein, and wherein R² is a nitrogen-linked carboxylic acid-containing group coupled to the nucleoside by a phosphoramide binding moiety, In a more particular embodiment of the foregoing, said carboxylic acid-containing group comprises two carboxylic acid groups linked to the nitrogen atom of the phosphoramide binding moiety either directly or via a short alkylene chain such as 1, 2 or 3 methylene groups. In an even more particular embodiment of the foregoing, R² is N(CH₂COOH)₂.

In other particular preferred embodiments of the present invention (phosphate-modified nucleosides represented by the structural formula (A) or the structural formula (I)), with reference to the structural formula (V), Z is O; NH or NCH₃.

In yet other particular embodiments of the present invention (phosphate-modified nucleosides represented by the structural formula (A) or the structural formula (I)), R⁴ is COOH and R⁵ is CH₂—COOH or CH₂-imidazolyl.

In particular embodiments of the present invention (phosphate-modified nucleosides represented by the structural formula (A) or the structural formula (I)), an aryl group is a C₆ aryl (i.e. phenyl) group optionally substituted with one or more substituents independently selected from the group consisting of halogen, amino, trifluoromethyl, hydroxyl, sulfhydryl, nitro, C₁₋₆ alkoxy, trifluoro-methoxy, cyano and (CH₂)_{q}—COOR⁶, wherein R⁶ is hydrogen or C₁₋₆ alkyl or benzyl, and q is 0, 1 or 2. In a more particular embodiment of the foregoing said C₆ aryl (i.e. phenyl) group is substituted with 1, 2 or 3 (CH₂)_{q}—COOR⁶, wherein R⁶ is hydrogen or C₁₋₆ alkyl or benzyl, and q is selected from 0, 1, and 2.

In other particular embodiments of the present invention (phosphate-modified nucleosides represented by the structural formula (A) or the structural formula (I)), R³ is hydrogen and each aryl group may be a C₆ aryl (i.e. phenyl) group substituted with 1, 2 or 3 (CH₂)_{q}—COOR⁶, wherein R⁶ is hydrogen or benzyl or C₁₋₆ alkyl, and q is 0, 1 or 2.

In yet more particular embodiments of the present invention (phosphate-modified nucleosides represented by the structural formula (A) or the structural formula (I)), R³ is hydrogen and each aryl group may be a C₆ aryl (i.e. phenyl) group substituted with two carboxylic acid groups such as 1,2-dicarboxyphenyl or 1,3-dicarboxyphenyl.

In yet more particular embodiments of the present invention (phosphate-modified nucleosides represented by the structural formula (A) or the structural formula (I)), R³ is hydrogen and R² is represented by the structural formula (V), wherein Z is O, S, NH or NCH₃, and Ar is phthalic acid or isophthalic acid.

In other particular embodiments of the present invention (phosphate-modified nucleosides represented by the structural formula (A) or the structural formula (I)), R³ is hydrogen and R² is represented by the structural formula (II), wherein n is 1, R⁴ is COOH and R⁵ is CH₂—COOH.

In other particular embodiments of the present invention (phosphate-modified nucleosides represented by the structural formula (A) or the structural formula (I)), R³ is hydrogen and R² is represented by the structural formula (II), wherein n is 1, R⁴ is COOCH₃ and R⁵ is CH₂—COOCH₃.

In a particular embodiment of the foregoing, the present invention relates to phosphate-modified nucleoside represented by the structural formula (I) wherein the pyrimidine analogue B is represented by the structural formula (C):

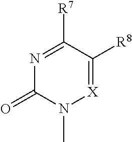

wherein
R⁷ is selected from the group consisting of —OH, —SH, —NH₂, —NHCH₃ and —NHC₂H₅;
R⁸ is selected from the group consisting of hydrogen, methyl, ethyl, isopropyl, amino, ethylamino, trifluoromethyl, cyano and halogen; and
X is CH or N.

In yet another particular embodiment of the foregoing, the present invention relates to phosphate-modified nucleosides represented by the structural formula (I) wherein the purine analogue is represented by the structural formula (D):

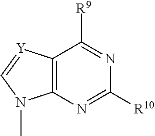

wherein
R⁹ is selected from the group consisting of H, —OH, —SH, —NH₂, and —NHCH₃;
R¹⁰ is selected from the group consisting of hydrogen, methyl, ethyl, hydroxyl, amino and halogen; and
Y is CH or N.

In a particular embodiment of the present invention, the novel phosphate-modified nucleoside may be selected from the group consisting of:

2′-deoxy-adenosine-5′-iminodiacetate-phosphoramidate (IA-dAMP); 2′-deoxy-cytidine-5′-iminodiacetate-phosphoramidate (IA-dCMP); 2′-deoxy-guanosine-5′-iminodiacetate-phosphoramidate (IA-dGMP); 2′-deoxy-thymidine-5′-iminodiacetate-phosphoramidate (IA-dTMP); and 2′-deoxy-uridine-5′-iminodiacetate-phosphoramidate (IA-dUMP), i.e. structural formulae (II) and (III) wherein R⁶ is hydrogen, n=1 and m=1;

2′-deoxy-adenosine-5′-iminodipropionate-phosphoramidate (IP-dAMP); 2′-deoxy-cytidine-5′-iminodipropionate-phosphoramidate (IP-dCMP); 2′-deoxy-guanosine-5′-iminodipropionate-phosphoramidate (IP-dGMP); 2′-deoxy-thymidine-5′-imino-dipropionate-phosphoramidate (IP-dTMP) and 2′-deoxy-uridine-5′-iminodipropionate-phosphoramidate (IP-dUMP), i.e. structural formulae (II) and (III) wherein R⁶ is hydrogen, n=2 and m=2; and

2′-deoxy-adenosine-5′-iminodibutyrate-phosphoramidate (IB-dAMP); 2′-deoxy-cytidine-5′-iminodibutyrate-phosphoramidate (IB-dCMP); 2′-deoxy-guanosine-5′-iminodibutyrate-phosphoramidate (IB-dGMP); 2′-deoxy-thymidine-5′-imino-dibutyrate-phosphoramidate (IB-dTMP) and 2′-deoxy-uridine-5′-iminodibutyrate-phosphoramidate (IB-dUMP), i.e. structural formulae (II) and (III) wherein R⁶ is hydrogen, n=3 and m=3.

In another particular embodiment of the present invention, the novel phosphate-modified nucleoside may be selected from the group consisting of:

adenosine-5′-iminodiacetate-phosphoramidate (IA-AMP); cytidine-5′-iminodiacetate-phosphoramidate (IA-CMP); guanosine-5′-iminodiacetate-phosphoramidate (IA-GMP); 5-methyluridine-5′-iminodiacetate-phosphoramidate (IA-m5 uMP), thymidine-5′-iminodiacetate-phosphoramidate (IA-TMP) and uridine-5′-iminodiacetate-phosphoramidate (IA-UMP), i.e. structural formulae (II) and (III) wherein R⁶ is hydrogen, n=1 and m=1;

adenosine-5′-iminodipropionate-phosphoramidate (IP-AMP); cytidine-5′-iminodipropionate-phosphoramidate (IP-CMP); guanosine-5′-iminodipropionate-phosphoramidate (IP-GMP); 5-methyluridine-5′-iminodipropionate-phosphoramidate (IP-m5 uMP), thymidine-5′-iminodipropionate-phosphoramidate (IP-TMP) and uridine-5′-iminodipropionate-phosphoramidate (IP-UMP), i.e. structural formulae (II) and (III) wherein R⁶ is hydrogen, n=2 and m=2; and

adenosine-5′-iminodibutyrate-phosphoramidate (IB-AMP); cytidine-5′-iminodibutyrate-phosphoramidate (IB-CMP); guanosine-5′-iminodibutyrate-phosphoramidate (IB-GMP); 5-methyluridine-5′-iminodibutyrate-phosphoramidate (IB-m5 uMP), thymidine-5′-iminodibutyrate-phosphoramidate (IB-TMP) and uridine-5′-iminodibutyrate-phosphoramidate (IB-UMP); i.e. structural formulae (II) and (III) wherein R⁶ is hydrogen, n=3 and m=3.

In another particular embodiment of the present invention, the novel phosphate-modified nucleoside may be selected from the group consisting of:

2′,3′-didehydro-2′,3′-dideoxy-adenosine-5′-iminodiacetate-phosphoramidate (IA-ddAMP); 2′,3′-didehydro-2′,3′-dideoxy-cytidine-5′-iminodiacetate-phosphoramidate (IA-ddCMP); 2′,3′-didehydro-2′,3′-dideoxy-guanosine-5′-iminodiacetate-phosphoramidate (IA-ddGMP); 2′,3′-didehydro-2′,3′-dideoxy-thymidine-5′-iminodiacetate-phosphoramidate (IA-ddTMP); and 2′,3′-didehydro-2′,3′-dideoxy-uridine-5′-iminodiacetate-phosphoramidate (IA-ddUMP), i.e. structural formulae (II) and (III) wherein R⁶ is hydrogen, n=1 and m=1;

2′,3′-didehydro-2′3′-dideoxy-adenosine-5′-iminodipropionate-phosphoramidate (IP-ddAMP); 2′,3′-didehydro-2′3′-dideoxy-cytidine-5′-iminodipropionate-phosphoramidate (IP-ddCMP); 2′,3′-didehydro-2′,3′-dideoxy-guanosine-5′-iminodipropionate-phosphoramidate (IP-ddGMP); 2′,3′-didehydro-2′3′-dideoxy-thymidine-5′-iminodipropionate-phosphoramidate (IP-ddTMP) and 2′,3′-didehydro-2′,3′-dideoxy-uridine-5′-iminodipropionate-phosphoramidate (IP-ddUMP), i.e. structural formulae (II) and (III) wherein R⁶ is hydrogen, n=2 and m=2; and

2′,3′-didehydro-2′,3′-dideoxy-adenosine-5′-iminodibutyrate-phosphoramidate (IB-ddAMP); 2′,3′-didehydro-2′,3′-dideoxy-cytidine-5′-iminodibutyrate-phosphoramidate (IB-ddCMP); 2′,3′-didehydro-2′,3′-dideoxy-guanosine-5′-iminodibutyrate-phosphoramidate (IB-ddGMP); 2′,3′-didehydro-2′,3′-dideoxy-thymidine-5′-imino-dibutyrate-phosphoramidate (IB-ddTMP) and 2′,3′-didehydro-2′,3′-deoxy-uridine-5′-iminodibutyrate-phosphoramidate (IB-ddUMP), i.e. structural formulae (II) and (III) wherein R⁶ is hydrogen, n=3 and m=3.

In yet another particular embodiment of the present invention, the novel phosphate-modified nucleoside may be selected from the group consisting of:

0.2′-deoxy-adenosine-5′-(dimethyl iminodiacetate)-phosphoramidate; 2′-deoxy-cytidine-5′-(dimethyl iminodiacetate)-phosphoramidate; 2′-deoxy-guanosine-5′-(dimethyl iminodiacetate)-phosphoramidate; 2′-deoxy-thymidine-5′-(dimethyl iminodiacetate)-phosphoramidate; 2′-deoxy-uridine-5′-(dimethyl iminodiacetate)-phosphoramidate, adenosine-5′-(dimethyl iminodiacetate)-phosphoramidate; cytidine-5′-(dimethyl iminodiacetate)-phosphoramidate; guanosine-5′-(dimethyl iminodiacetate)-phosphoramidate; thymidine-5′-(dimethyl iminodiacetate)-phosphoramidate; uridine-5′-(dimethyl iminodiacetate)-phosphoramidate; 2′,3′ didehydro-2′,3′-dideoxy-adenosine-5′-(dimethyl iminodiacetate)-phosphoramidate; 2′,3′ didehydro-2′,3′-dideoxy-cytidine-5′-(dimethyl iminodiacetate)-phosphoramidate; 2′,3′ didehydro-2′,3′-dideoxy-guanosine-5′-(dimethyl iminodiacetate)-phosphoramidate; 2′,3′ didehydro-2′,3′-dideoxy-thymidine-5′-(dimethyl iminodiacetate)-phosphoramidate; and 2′,3′ didehydro-2′,3′-dideoxy-uridine-5′-(dimethyl iminodiacetate)-phosphoramidate, i.e. structural formulae (II) and (III) wherein R⁶ is methyl, n=1 and m=1;

2′-deoxy-adenosine-5′-(diethyl iminodiacetate)-phosphoramidate; 2′-deoxy-cytidine-5′-(diethyl iminodiacetate)-phosphoramidate; 2′-deoxy-guanosine-5′-(diethyl iminodiacetate)-phosphoramidate; 2′-deoxy-thymidine-5′-(diethyl iminodiacetate)-phosphoramidate; 2′-deoxy-uridine-5′-(diethyl iminodiacetate)-phosphoramidate, adenosine-5′-(diethyl iminodiacetate)-phosphoramidate; cytidine-5′-(diethyl iminodiacetate)-phosphoramidate; guanosine-5′-(diethyl iminodiacetate)-phosphoramidate; thymidine-5′-(diethyl iminodiacetate)-phosphoramidate; uridine-5′-(diethyl iminodiacetate)-phosphoramidate; 2′,3′ didehydro-2′,3′-dideoxy-adenosine-5′-(diethyl iminodiacetate)-phosphoramidate; 2′,3′ didehydro-2′,3′-dideoxy-cytidine-5′-(diethyl iminodiacetate)-phosphoramidate; 2′,3′ didehydro-2′,3′-dideoxy-guanosine-5′-(diethyl iminodiacetate)-phosphoramidate; 2′,3′ didehydro-2′,3′-dideoxy-thymidine-5′-(diethyl iminodiacetate)-phosphoramidate; and 2′,3′ didehydro-2′,3′-dideoxy-uridine-5′-(diethyl iminodiacetate)-phosphoramidate, i.e. structural formulae (II) and (III) wherein R⁶ is ethyl, n=1 and m=1;

2′-deoxy-adenosine-5′-(dibenzyl iminodiacetate)-phosphoramidate; 2′-deoxy-cytidine-5′-(dibenzyl iminodiacetate)-phosphoramidate; 2′-deoxy-guanosine-5′-(dibenzyl iminodiacetate)-phosphoramidate; 2′-deoxy-thymidine-5′-(dibenzyl iminodiacetate)-phosphoramidate; 2′-deoxy-uridine-5′-(dibenzyl iminodiacetate)-phosphoramidate, adenosine-5′-(dibenzyl iminodiacetate)-phosphoramidate; cytidine-5′-(dibenzyl iminodiacetate)-phosphoramidate; guanosine-5′-(dibenzyl iminodiacetate)-phosphoramidate; thymidine-5′-(dibenzyl iminodiacetate)-phosphoramidate; uridine-5′-(dibenzyl iminodiacetate)-phosphoramidate; 2′,3′ didehydro-2′,3′-dideoxy-adenosine-5′-(dibenzyl iminodiacetate)-phosphoramidate; 2′,3′ didehydro-2′,3′-dideoxy-cytidine-5′-(dibenzyl iminodiacetate)-phosphoramidate; 2′,3′ didehydro-2′,3′-dideoxy-guanosine-5′-(dibenzyl iminodiacetate)-phosphoramidate; 2′,3′ didehydro-2′,3′-dideoxy-thymidine-5′-(dibenzyl iminodiacetate)-phosphoramidate; and 2′,3′ didehydro-2′,3′-dideoxy-uridine-5′-(dibenzyl iminodiacetate)-phosphoramidate, i.e. structural formulae (II) and (III) wherein R⁶ is benzyl, n=1 and m=1;

0.2′-deoxy-adenosine-5′-(dimethyl iminodipropionate)-phosphoramidate; 2′-deoxy-cytidine-5′-(dimethyl iminodipropionate)-phosphoramidate; 2′-deoxy-guanosine-5′-(dimethyl iminodipropionate)-phosphoramidate; 2′-deoxy-thymidine-5′-(dimethyl iminodipropionate)-phosphoramidate; 2′-deoxy-uridine-5′-(dimethyl iminodipropionate)-phosphoramidate, adenosine-5′-(dimethyl iminodipropionate)-phosphoramidate; cytidine-5′-(dimethyl iminodipropionate)-phosphoramidate; guanosine-5′-(dimethyl iminodipropionate)-phosphoramidate; thymidine-5′-(dimethyl iminodipropionate)-phosphoramidate; uridine-5′-(dimethyl iminodipropionate)-phosphoramidate; 2′,3′ didehydro-2′,3′-dideoxy-adenosine-5′-(dimethyl iminodipropionate)-phosphoramidate; 2′,3′ didehydro-2′,3′-dideoxy-cytidine-5′-(dimethyl iminodipropionate)-phosphoramidate; 2′,3′ didehydro-2′,3′-dideoxy-guanosine-5′-(dimethyl iminodipropionate)-phosphoramidate; 2′,3′ didehydro-2′,3′-dideoxy-thymidine-5′-(dimethyl iminodipropionate)-phosphoramidate; and 2′,3′ didehydro-2′,3′-dideoxy-uridine-5′-(dimethyl iminodipropiontate)-phosphoramidate, i.e. structural formulae (II) and (III) wherein R⁶ is methyl, n=2 and m=2;

2′-deoxy-adenosine-5′-(diethyl iminodipropionate)-phosphoramidate; 2′-deoxy-cytidine-5′-(diethyl iminodipropionate)-phosphoramidate; 2′-deoxy-guanosine-5′-(diethyl iminodipropionate)-phosphoramidate; 2′-deoxy-thymidine-5′-(diethyl iminodipropionate)-phosphoramidate; 2′-deoxy-uridine-5′-(diethyl iminodipropionate)-phosphoramidate, adenosine-5′-(diethyl iminodipropionate)-phosphoramidate; cytidine-5′-(diethyl iminodipropionate)-phosphoramidate; guanosine-5′-(diethyl iminodipropionate)-phosphoramidate; thymidine-5′-(diethyl iminodipropionate)-phosphoramidate; uridine-5′-(diethyl iminodipropionate)-phosphoramidate; 2′,3′ didehydro-2′,3′-dideoxy-adenosine-5′-(diethyl iminodipropionate)-phosphoramidate; 2′,3′ didehydro-2′,3′-dideoxy-cytidine-5′-(diethyl iminodipropionate)-phosphoramidate; 2′,3′ didehydro-2′,3′-dideoxy-guanosine-5′-(diethyl iminodipropionate)-phosphoramidate; 2′,3′ didehydro-2′,3′-dideoxy-thymidine-5′-(diethyl iminodipropionate)-phosphoramidate; and 2′,3′ didehydro-2′,3′-dideoxy-uridine-5′-(diethyl iminodipropionate)-phosphoramidate, i.e. structural formulae (II) and (III) wherein R⁶ is ethyl, n=2 and m=2;

In another particular embodiment of the present invention, the novel phosphate-modified nucleoside may be selected from the groups consisting of 5-O-IsoPhthalicAcid-dAMP, 5-NH-IsoPhthalicAcid-dAMP, 4-O-PhthalicAcid-dAMP, 5-O-IsoPhthalicAcid-dCMP, 5-NH-IsoPhthalicAcid-dCMP, 4-O-PhthalicAcid-dCMP, 5-O-IsoPhthalicAcid-dGMP, 5-NH-IsoPhthalicAcid-dGMP, 4-O-PhthalicAcid-dGMP, 5-O-IsoPhthalicAcid-dTMP, 5-NH-IsoPhthalicAcid-dTMP, 4-O-PhthalicAcid-dTMP, 5-O-IsoPhthalicAcid-dUMP, 5-NH-IsoPhthalicAcid-dUMP, or 4-O-PhthalicAcid-dUMP (in each of these abbreviated names, the abbreviation “phthalic acid” reads for the more standard notation “1,2-dicarboxyphenyl” and the abbreviation “isophthalic acid” reads for the more standard notation “1,3-dicarboxyphenyl”).

In another particular embodiment of the present invention, the novel phosphate-modified nucleoside may be selected from the groups consisting of 5-O-IsoPhthalicAcid-AMP, 5-NH-IsoPhthalicAcid-AMP, 4-O-PhthalicAcid-AMP, 5-O-IsoPhthalicAcid-CMP, 5-NH-IsoPhthalicAcid-CMP, 4-O-PhthalicAcid-CMP, 5-O-IsoPhthalicAcid-GMP, 5-NH-IsoPhthalicAcid-GMP, 4-O-PhthalicAcid-GMP, 5-O-IsoPhthalicAcid-TMP, 5-NH-IsoPhthalicAcid-TMP, 4-O-PhthalicAcid-TMP, 5-O-IsoPhthalicAcid-UMP, 5-NH-IsoPhthalicAcid-UMP, or 4-O-PhthalicAcid-UMP (in each of these abbreviated names, the abbreviation “phthalic acid” reads for the more standard notation “1,2-dicarboxyphenyl” and the abbreviation “isophthalic acid” reads for the more standard notation “1,3-dicarboxyphenyl”).

Another aspect of the present invention relates to the use of the phosphate-modified nucleosides represented by the structural formulae (A) and (I), including any one of the above-referred specific embodiments thereof, as a substrate for DNA- or RNA-polymerases, these polymerases being either wild-type (naturally occurring) or mutated according to common knowledge in the art. In a particular embodiment, said DNA- or RNA-polymerases are selected from Therminator DNA polymerase, KF (exo⁻) DNA polymerase, or Reverse Transcriptase (e.g. HIV-RT) or mutated forms of these enzymes. If needed, the enzymes as described herein above can be mutated, using common knowledge in the art, in order to better adapt to the novel phosphate-modified nucleoside disclosed in this invention. In a particular embodiment, the present invention relates to the use of the phosphate-modified nucleosides of the invention, as a substrate for DNA- or RNA-polymerases in bacteriae or in vitro. In another particular embodiment, said DNA- or RNA-polymerase originates from a micro-organism or from bacterial or viral origin.

In a particular embodiment, the phosphate-modified nucleosides of this invention being represented by the structural formulae (A) and (I), including any one of the above-referred specific embodiments thereof, can be used to build in at least 1, 2 or 3 nucleotides in a growing DNA- or RNA-strand.

In another particular embodiment, the phosphate-modified nucleosides of this invention being represented by the structural formulae (A) and (I), including any one of the above-referred specific embodiments thereof, can be used to build in at most 1, 2 or 3 nucleotides in a growing DNA- or RNA-strand.

In another particular embodiment, the phosphate-modified nucleosides of this invention being represented by the structural formulae (A) and (I), including any one of the above-referred specific embodiments thereof, can be used to build in at most 300 nucleotides in a growing DNA- or RNA-strand.

In yet another particular embodiment, the phosphate-modified nucleosides of this invention being represented by the structural formulae (A) and (I), including any one of the above-referred specific embodiments thereof, can be used with a mixture of natural dNTPs or NTPs (ATP,CTP,GTP,UTP,TTP) as a substrate for DNA/RNA-polymerases, more in particular to build in 1-300 (e.g. 2-300) nucleotides in a growing DNA- or RNA-strand.

The present invention also relates to the use of the phosphate-modified nucleoside represented by the structural formulae (A) and (I), including any one of the above-referred specific embodiments thereof, for the enzymatic production of oligonucleotides, peptides or proteins.

In a particular embodiment, the phosphate-modified nucleosides of the invention being represented by the structural formulae (A) and (I), including any one of the above-referred specific embodiments thereof, can be used for in vitro production of DNA or RNA. In another particular embodiment, the phosphate-modified nucleosides of the invention being represented by the structural formulae (A) and (I), including any one of the above-referred specific embodiments thereof, can also be used for in vitro production of peptides or proteins. In another particular embodiment the phosphate-modified nucleosides of the invention being represented by the structural formulae (A) and (I), including any one of the above-referred specific embodiments thereof, can be used for PCR (polymerase chain reaction).

In yet another particular embodiment, the phosphate-modified nucleosides of the invention being represented by the structural formulae (A) and (I), including any one of the above-referred specific embodiments thereof, can be used as a substrate for the growth of wild type and/or mutated bacteriae. In a particular embodiment, the phosphate-modified nucleotides of the invention being represented by the structural formulae (A) and (I), including any one of the above-referred specific embodiments thereof, can be used as a substrate for the growth of bacteriae with mutated DNA/RNA polymerase, preferably wherein the mutation is suitable to better adapt better to the new substrate.

In view of their antiviral activity discussed below, another aspect of the present invention relates to a pharmaceutical, veterinary or non-pharmaceutical composition comprising an anti-virally effective amount of a phosphate-modified nucleoside of the invention being represented by the structural formulae (A) and (I), including any one of the above-referred specific embodiments thereof. In a particular embodiment, said pharmaceutical, veterinary or non-pharmaceutical composition may further comprise an aqueous solution and optionally one or more buffering agents. In a particular embodiment, said pharmaceutical, veterinary or non-pharmaceutical composition may further comprise one or more natural NTPs or dNTPs (e.g. ATP, CTP, GTP, UTP or TTP).

Another aspect of the invention relates to the use of the non-pharmaceutical composition of the invention as a substrate to build in at least 1, 2 or 3 nucleotides in a growing DNA- or RNA-strand.

Yet another aspect of the invention relates to the use of the phosphate-modified nucleosides of the invention, being represented by the structural formulae (A) and (I), including any one of the above-referred specific embodiments thereof, in a non-human living organism for sustaining growth, survival or proliferation of said organism. In a particular embodiment said organism is selected from the group consisting of a virus, a bacterium, an archaeon and an eukaryote, and in a more particular embodiment said eukaryote is selected from the group consisting of yeast, mold, fungus, microalga, multicellular plant and protist.

Yet another aspect of the invention relates to a method for the production of oligonucleotides, RNA, DNA, peptides and/or proteins by using the phosphate-modified nucleotides of the invention, being represented by the structural formulae (A) and (I), including any one of the above-referred specific embodiments thereof.

Another aspect of the present invention relates to compounds represented by the structural formulae (A) and (I), including any one of the above-referred specific embodiments thereof, having antiviral activity, specifically to these compounds that inhibit the replication of viruses (such as, but not limited to, viruses belonging to the order Herpesvirales, in particular the family Herpesviridae, the family Alloherpesviridae or the family Malacoherpesviridae), in particular retroviruses, and even more specifically to these compounds that inhibit the replication of HIV-1 or HIV-2.

Another aspect of the present invention relates to the compounds represented by the structural formulae (A) and (I), including any one of the above-referred specific embodiments thereof, for use as a medicine, more particularly for use to treat or prevent a viral infection in a mammal, even more particularly to treat or prevent HIV infection in a mammal such as a human being.

Another aspect of the invention relates to pharmaceutical compositions comprising an anti-virally effective amount of at least one compound being represented by the structural formulae (A) and (I), including any one of the above-referred specific embodiments thereof, in combination with one or more pharmaceutically acceptable excipients being well known in the art for the formulation of phosphate nucleosides. The invention further relates to the use of the compounds represented by the structural formulae (A) and (I), including any one of the above-referred specific embodiments thereof, in the manufacture of a medicament useful for the treatment of viral infections (e.g. from a virus belonging to the order Herpesvirales), more specifically for the treatment of a retroviral infection such as a HIV-1 or HIV-2 infection.

The present invention also relates to a method of treatment or prevention of a viral infection in a mammal, comprising the administration of a therapeutically effective (e.g. anti-virally effective or replication-inhibiting) amount of a compound of this invention represented by the structural formulae (A) and (I), including any one of the above-referred specific embodiments thereof, optionally in combination with one or more pharmaceutically acceptable excipients. In a more particular embodiment of the foregoing, said viral infection is a HIV infection. In a more particular embodiment of the foregoing, said mammal is a human being.

Still another aspect of the invention relates to processes and methods for the preparation of the phosphate-modified nucleosides of the invention represented by the structural formulae (A) and (I), including any one of the above-referred specific embodiments thereof. In one embodiment of said aspect, the method comprises the steps of interacting a nucleoside monophosphate (NMP) with an ester, e.g. a methyl, ethyl or benzyl ester of the carboxylic acid to be introduced as a leaving group into the compound of this invention to produce the ester of a phosphoramidate nucleoside analogue, as depicted in any one of schemes 2, 3, 4 and 5 below. Deprotection of this nucleoside analogue with a deprotecting agent such as, but not limited to, an alkali hydroxide, e.g. 0.04 M NaOH, provides the desired phosphoramidate nucleoside of this invention.

In an alternative embodiment of said aspect, the process for the preparation of the phosphate-modified nucleosides of the invention comprises a synthetic step as shown in the following scheme 1:

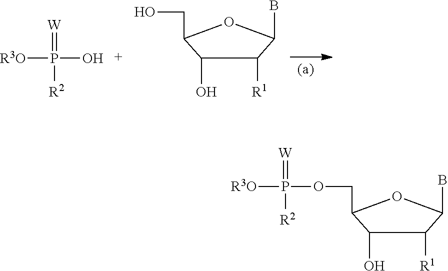

wherein (a) schematically represents the presence in the reaction mixture of an effective amount of a suitable catalyst for the condensation of the 5′-OH and phosphate acid groups. Suitable such catalysts are well known in the art and include, but are not limited to, an arylsulfonyl halide, e.g. an optionally substituted phenylsulfonyl chloride. Phosphates, phosphorothioates and phosphoramidates wherein R² and R³ are as defined in the structural formulae (A) and (I), including any one of the above-referred specific embodiments thereof, as shown on the left part of scheme 1 to be used as starting materials of this method may be known in the art or may be produced according to one or more of the synthetic procedures as described by Scheit in Nucleotide analogs, J Wiley and sons, New York (1980) or by Vaghefi in Nucleoside Triphosphate and their analogs, Taylor and Francis, CRC Press (2005), the content of which is incorporated by reference.

### BRIEF DESCRIPTION OF THE DRAWINGS

FIG. 1 schematically shows a synthetic procedure for producing a modified nucleoside according to one embodiment of the present invention including a phosphoramidate moiety wherein R² and R⁵ are represented by the structural formulae (II) and (III) respectively, and R⁴ and R¹¹ are both carboxylic acid groups.

FIG. 2 shows a representation of the chemical structures of 5-O-isophthalic acid-dAMP 2,5-NH-isophthalic acid-dAMP 3, and 4-O-phthalic acid-dAMP 4 respectively.

FIG. 3 shows the incorporation of the illustrative compounds 25-O-iPA-dAMP, 3 5-NH-iPA-dAMP, and 4 4-O-PA-dAMP of this invention into P1T1 by HIV Reverse Transcriptase. Aliquots were taken at 5, 10, 20, 30, 60 and 120 minutes, indicated as 1, 2, 3, 4, 5 and 6 respectively. For dATP, aliquots were taken at 5, 30, 60 and 120 minutes, indicated as 1, 2, 3 and 4 respectively.

FIG. 4 shows the incorporation of 5-H-iPA-dAMP (compound 2) at different concentrations into P1T1 by HIV Reverse Transcriptase. Aliquots were taken at 5, 10, 20, 30, 60 and 120 minutes, indicated as 1, 2, 3, 4, 5 and 6 respectively. For dATP, aliquots were taken at 5, 10, 20, 30 and 60 minutes, indicated as 1, 2, 3, 4 and 5 respectively.

FIG. 5 shows the elongation of P1T2 with IA-dAMP by HIV-1 Reverse Transcriptase. Aliquots were taken at 15, 30, 60, 90 and 120 minutes, indicated as 1, 2, 3, 4 and 5 respectively. No triphosphate analogue was added in the blank reaction.

FIG. 6 shows the elongation of P1T3 with IA-dAMP by HIV-1 Reverse Transcriptase. Aliquots were taken at 15, 30, 60, 90 and 120 minutes, indicated as 1, 2, 3, 4 and 5 respectively. No triphosphate analogue was added in the blank reaction.

FIGS. 7A and 7B respectively show the incorporation of the illustrative compounds 5 and 6 of this invention into P1T1 by HIV-1 Reverse Transcriptase.

FIGS. 8 and 9 show the data of elongation capacity of P1T2 with the illustrative compound 5 of this invention by HIV-1 Reverse Transcriptase.

FIG. 10 shows activation of AZT monophosphate by a novel leaving group moiety of this invention (top) and an application of iminodiacetate phosphoramidate moiety for in vivo delivery and activation of AZT monophosphate (bottom).

### DEFINITIONS

The term “nucleoside” conventionally refers to natural glycosylamines consisting of a nucleobase bound to a ribose or deoxyribose sugar via a β-glycosidic linkage such as cytidine, urisine, adenosine, guanosine and thymidine. The term “nucleoside analogue” as used herein refers to known nucleosides consisting of a sugar linked to a pyrimidine or purine base, including modifications wherein the sugar ring is modified and/or wherein the nucleobase is further substituted. Nucleoside analogues wherein the natural sugar moiety is modified include but are not limited to:

nucleoside analogues wherein the ribose sugar is replaced with another monocyclic sugar such as, but not limited to, arabinofuranose, arabinopyranose, xylofuranose, xylopyranose, lyxofuranose, lyxopyranose, α-D-threofuranose; threose nucleic acids (TNA) also referred as α-threofuranosyl nucleosides are described for example by Orgel in Science 290 (5495) 1306-1307 and by Schong et al in Science 290 (5495) 1347-1351, the content of which is incorporated herein by reference;

nucleoside analogues wherein the ribose sugar is replaced with a bicyclic or tricyclic sugar, such as locked nucleic acids (LNA) wherein the ribose moiety is modified with at least an extra bridge connecting the 2′-oxygen and 4′-carbon atoms, wherein the bridge locks the ribose in the 3′-endo conformation; LNA are described for example in WO 99/14226, the content of which is incorporated herein by reference;

nucleoside analogues wherein the (deoxy)ribose sugar is unsaturated (dehydrodeoxyribose) or substituted with one or more conventional substituents (e.g. azido) for nucleoside technology such as, but not limited to, 2′,3′-deoxy-3′-azidoribose.

The term “sugar” as used herein includes ribose and deoxyribose linked by the oxygen atom at position 5 of the ribose or deoxyribose moiety to the phosphorus atom P in the structural formula (A) or the structural formula (I), but is not limited to, i.e. also includes modifications and variants of the ribose or deoxyribose moiety. Such modifications are well known to those skilled in the art, examples being pentofuranoses such as listed above, unsaturated and/or substituted monocyclic sugars such as, but not limited to, 2,3-deoxy-3-azido-ribose, and also bicyclic or tricyclic sugars such as present in locked nucleic acids (LNA).

The term “pyrimidine or purine base” as used herein includes, but is not limited to, adenine, thymine, cytosine, uracyl, guanine and 2,6-diaminopurine and analogues and derivatives thereof. A purine or pyrimidine base as used herein includes a purine or pyrimidine base found in naturally occurring nucleosides as mentioned above. An analogue thereof is a base which mimics such naturally occurring bases in such a way that their structures (the kinds of atoms and their arrangement) are similar to the naturally occurring bases but may either possess additional or lack certain of the functional properties of the naturally occurring bases. Such analogues include those derived by replacement of a CH moiety by a nitrogen atom (e.g. 5-azapyrimidines such as 5-azacytosine) or vice versa (e.g., 7-deazapurines, such as 7-deazaadenine or 7-deazaguanine) or both (e.g., 7-deaza, 8-azapurines). By derivatives of such bases or analogues are meant those bases wherein one or more ring substituents are either incorporated, removed, or modified by conventional substituents known in the art, e.g. halogen, hydroxyl, amino, C₁₋₆ alkyl and other non-reactive and biocompatible substituents. Such purine or pyrimidine bases, and analogues and derivatives thereof, are well known to those skilled in the art, e.g. as shown at pages 20-38 of WO 03/093290, Horlacher et al. in PNAS (1995) 92:6329 and U.S. Pat. No. 6,617,106 (specifically on pages 3-5), the content of which is incorporated herein by reference.

In particular purine and pyrimidine analogues B for the purpose of the present invention may be selected from the group comprising pyrimidine bases represented by the structural formula (C):

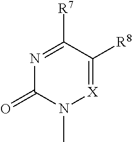

and purine bases and analogues represented by the structural formula (D):

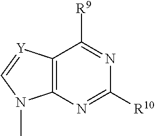

wherein:
R⁷ and R⁹ are independently selected from the group consisting of H, —OH, —SH, —NH₂, and —NH-Me;
R⁸ and R¹⁹ are independently selected from the group consisting of H, methyl, ethyl, isopropyl, hydroxyl, amino, ethylamino, trifluoromethyl, cyano and halogen; and
X and Y are independently selected from CH and N.

Just as a few non-limiting examples of pyrimidine analogues, can be named substituted uracils with the formula (C) wherein X is CH, R⁷ is hydroxyl, and R⁸ is selected from the group consisting of methyl, ethyl, isopropyl, amino, ethylamino, trifluoromethyl, cyano, fluoro, chloro, bromo and iodo.

The term “C₁₋₆ alkyl” as used herein refers to normal, secondary, or tertiary hydrocarbon chains having from 1 to 6 carbon atoms. Examples thereof are methyl, ethyl, 1-propyl, 2-propyl, 1-butyl, 2-methyl-1-propyl(i-Bu), 2-butyl(s-Bu) 2-methyl-2-propyl(t-Bu), 1-pentyl(n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl.

As used herein and unless otherwise stated, the term “cycloalkyl” means a monocyclic saturated hydrocarbon monovalent group having from 3 to 10 carbon atoms, such as for instance cyclopropyl, cyclobutyl, cyclopentyl, cyclohexyl, cycloheptyl, cyclooctyl, or a C₇₋₁₀ polycyclic saturated hydrocarbon monovalent group having from 7 to 10 carbon atoms such as, for instance, norbornyl, fenchyl, trimethyltricycloheptyl or adamantyl.

The term “C₁₋₆ alkoxy” as used herein refers to substituents wherein a carbon atom of a C₁₋₆ alkyl group (such as defined herein), is attached to an oxygen atom through a single bond such as, but not limited to, methoxy, ethoxy, propoxy, isopropoxy, butoxy, iso-butoxy, sec-butoxy, tert-butoxy, pentoxy, 3-pentoxy, or n-hexyloxy.

As used herein, and unless otherwise stated, the term “aryl” designates any mono- or polycyclic aromatic monovalent hydrocarbon group having from 6 up to 30 carbon atoms such as, but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including benzo-fused cycloalkyl radicals (the latter being as defined above) such as, for instance, indanyl, tetrahydronaphthyl, fluorenyl and the like, all of the said groups being optionally substituted with one or more substituents independently selected from the group consisting of halogen, amino, trifluoromethyl, hydroxyl, sulfhydryl, nitro, C₁₋₆ alkoxy, trifluoromethoxy, cyano and (CH₂)_{q}—COOR⁶, wherein R⁶ is selected from the group consisting of hydrogen, C₁₋₆ alkyl and benzyl, and q is selected from 0, 1, and 2, such as for instance carboxyphenyl, phthalic acid (1,2-dicarboxyphenyl), isophthalic acid (1,3-dicarboxyphenyl), 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein with respect to a substituting group, and unless otherwise stated, the term “aryl-C₁₋₆ alkyl” refers to an aliphatic saturated hydrocarbon monovalent group (preferably a C₁₋₆ alkyl group such as defined above) onto which an aryl group (such as defined above) is linked via a carbon atom, and wherein the said aliphatic group and/or the said aryl group may be optionally substituted with one or more substituents independently selected from the group consisting of halogen, amino, hydroxyl, sulfhydryl, C₁₋₆ alkyl, C₁₋₆ alkoxy, trifluoromethyl, trifluoromethoxy, nitro and carboxylic acid, such as but not limited to benzyl, 4-chlorobenzyl, 4-fluorobenzyl, 2-fluorobenzyl, 3,4-dichlorobenzyl, 2,6-dichlorobenzyl, 3-methylbenzyl, 4-methylbenzyl, 4-tert-butylbenzyl, phenylpropyl, 1-naphthylmethyl, phenylethyl and the like.

As used herein and unless otherwise stated, the term halogen means any atom selected from the group consisting of fluorine (F), chlorine (Cl), bromine (Br) and iodine (I).

Any substituent designation that is found in more than one place in a modified nucleoside of this invention may be independently selected at each place.

The term “amino acid” as used herein refers to any “natural amino acid” (Alanine (ala), Arginine (Arg), Asparagine (asn), Aspartic acid (Asp), Cysteine (cys), Glutamine (gin), Glutamic acid (glu), Glycine (gly), Histidine (his), Hydroxylysine (Hyl), Hydroxyproline (Hyp), Isoleucine (ile), Leucine (leu), Lysine (lys), Methionine (met), Phenylalanine (phe), Proline (pro), Serine (ser), Threonine (thr), Tryptophan (trp), Tyrosine (tyr), Valine (val)) in D or L conformation (but, within the context of this invention, preferably the L conformation), as well as to “unnatural (or synthetic) amino acids” (e.g., but not limited to, phosphoserine, phosphothreonine, phosphotyrosin, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, alpha-methyl-alanine, para-benzoylphenylalanine, phenylglycine, propargylglycine, sarcosine, and tert-butylglycine). This term also comprises natural and non-natural amino acids being protected at their carboxylic terminus (e.g. as a C₁₋₆ alkyl, phenyl or benzyl ester or as an amide, such as for example, a mono-C₁₋₆alkyl or di-(C₁₋₆ alkyl) amide. Other suitable carboxy protecting groups are known to those skilled in the art (see for example, T. W. Greene, Protecting Groups In Organic Synthesis; Wiley: New York, (1981) and references cited therein, the content of which is incorporated herein by reference).

It will be appreciated by those skilled in the art that certain modified nucleosides of this invention having a chiral center may exist in, and be isolated in, optically active and racemic forms. Some modified nucleosides may exhibit polymorphism. It is to be understood that the present invention encompasses any racemic, optically-active, polymorphic, or stereoisomeric form, or mixtures thereof in any proportions, of a modified nucleoside of this invention, which may possess the useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase).

As used herein and unless otherwise stated, the term “stereoisomer” refers to all possible different isomeric as well as conformational forms which the compounds of formula (I) may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

Certain of the phosphate-modified nucleotides herein when substituted with appropriate selected functionalities are capable of acting as pro-drugs. These are labile functional groups which separate from an active inhibitory phosphate-modified nucleotide during metabolism, systemically, inside a cell, by hydrolysis, enzymatic cleavage, or by some other process (Bundgaard “Design and Application of Pro-drugs” in Textbook of Drug Design and Development (1991), Eds. Harwood Academic Publishers, pp. 113-191, the content of which is incorporated herein by reference). These pro-drug moieties can serve to enhance solubility, absorption and lipophilicity to optimize drug delivery, bioavailability and efficacy. A “pro-drug” is thus a covalently modified analogue of a therapeutically-active modified nucleoside of this invention. A pro-drug moiety can also be therapeutically active in its own right.

Exemplary suitable pro-drug moieties include, but are not limited to, esters of nucleosides like the POM (pivaloyloxymethyl), POC (isopropyloxycarbonyloxymethyl) and SATE (S-acyl-2-thioethyl) esters.

The term “salt” as used herein, refers to the ionic product of a reaction between an acid and a base. Salts of the compounds having structural formula I may be formed at any acid or base functionality within the compound, in particular, R³, R⁴ and R⁵ may represent or comprise an acid or base functionality. In particular, salts of the compounds represented by the structural formula (I) or the structural formula (A) may be formed as follows. When R³ is hydrogen, it is acidic and may therefore engage in salt formation with an inorganic or organic base. R⁴ and R⁵ comprise acid functionalities such as carboxylic groups (i.e. —COOH), which can equally engage in salt formation with an organic or inorganic base. Alternatively, R⁴ and R⁵ may comprise base functionalities such as the imidazolyl, which in turn can engage in salt formation with an organic or inorganic acid.

The term “pro-drug”, as used herein, relates to an inactive or active derivative of a compound represented by the structural formula (I) or the structural formula (A) as defined herein above or any one of their specific embodiments, which undergoes spontaneous or enzymatic transformation within the body of an animal, e.g. a mammal such as a human being, in order to release the pharmacologically active form of the compound. For a comprehensive review, reference is made to Rautio J. et al. (“Pro-drugs: design and clinical applications” in Nature Reviews Drug Discovery (2008) doi: 10.1038/nrd2468, the content of which is incorporated herein by reference). In particular for the purpose of the present invention, pro-drugs of the compounds represented by the structural formula (A) or the structural formula (I), including any one of the above-described specific embodiments thereof, may be formed as follows. When R³ is H, a free phosphate acid function is available for pro-drug formation as described in detail by Hecker et al. (“Prodrugs of phosphates and phosphonates” Journal of Medicinal Chemistry (2008) doi: 10.1021/jm701260b, the content of which is incorporated herein by reference). R⁴ and R⁵ comprise acid functionalities such as carboxylic acid groups (i.e. —COOH) which may be used for the formation of a pro-drug. Such carboxylic acid pro-drug may occur in the form of an ester, in particular acyloxyalkyl esters (e.g. pivaloyloxymethyl ester (POM)) or S-acylthioethyl (SATE) esters, a carbonate, a carbamate or an amide, such as amino acid pro-drugs.

The term “peptide” as used herein refers to a sequence of 2 to 50 amino acids (e.g. as defined hereinabove) or peptidyl residues. The sequence may be linear or cyclic. Preferably a peptide comprises 2 to 25, or 5 to 20 amino acids.

The term “oligonucleotide” as used herein refers to a polynucleotide formed by a plurality of linked nucleotide units. The nucleotide units each include a nucleoside unit linked together via a phosphate linking group. These nucleotides can be natural or modified in their phosphate, sugar or nucleobase group. The oligonucleotide may be naturally occurring or non naturally occurring.

The term “polymerase” as used herein refers to an enzyme that can synthesize DNA or RNA from a DNA or RNA template and includes but is not limited to Therminator DNA polymerase, KF(exo⁻)DNA polymerase and HIV Reverse Transcriptase.

### Biological Applications of the Invention

Novel phosphoramidates, phospho-esters and phospho-thioesters according to this invention may be used as alternative substrates and biotechnology tools.

Fast emerging applications of modified nucleosides as biotechnology tools also require new and efficient ways to synthesize DNA and RNA building blocks such as nucleoside triphosphates and amidites for the use, for example, in PCR, labeling, or enzymatic incorporation of nucleotides, and in the automated DNA synthesis, respectively. Furthermore, some biotechnology applications require incorporation of a nucleotide by enzymatic means using DNA or RNA polymerases. However, at times, due to chemical nature and modifications present in the modified nucleosides, triphosphate synthesis is not always feasible and/or provides insufficient and low yields.

Therefore, carboxyl-containing groups coupled to a nucleoside monophosphate through a phosphoramidate (P—N) bond can serve as an alternative or substitute group to a pyrophosphate moiety. However, fitting into an active site and the subsequent nucleotidyl transfer may be less efficient for such carboxyl-containing phosphoramidate (e.g. IA-dAMP) compared to the natural substrates/dNTPs (e.g. dATP) for the natural polymerase/enzyme. In this situation, mutated polymerases can be used to increase the efficiency of recognition and incorporation of the compounds of this invention. Such an embodiment of the invention with mutated polymerases can be used to specifically select or grow bacteriae by using these carboxyl-containing phosphoramidate nucleosides as a substrate. An additional advantage of this application is that polymerases that demonstrated efficient recognition and incorporation of carboxyl-containing phosphoramidate nucleosides in our studies are also shown to tolerate various sugar modifications and unnatural nucleobases quite well. Therefore, the enzymatic synthesis of DNA and, RNA sequences containing unnatural nucleobases can be accomplished whilst avoiding at times cumbersome nucleoside triphosphate synthesis and purification.

The phosphoramidates, phospho-esters and phospho-thioesters of this invention are also useful as antiviral compounds

The compounds of the invention can be efficiently used for the treatment of viral infections, particularly retroviral infections, more particularly Human Immunodeficiency Virus (HIV) infections, in particular of Human Immunodeficiency Virus type 1 (HIV-1). When using one or more derivatives represented by the structural formula (I) or the structural formula (A) as defined herein:

the active ingredients of the compound(s) may be administered to the mammal (including a human being) to be treated by any means well known in the art, i.e. orally, intranasally, subcutaneously, intramuscularly, intradermally, intravenously, intra-arterially, parenterally or by catheterization;

the therapeutically effective amount of the preparation of the compound(s), especially for the treatment of viral infections in humans and other mammals, preferably is a HIV enzyme inhibiting amount. More preferably, it is a HIV replication inhibiting amount or a HIV enzyme (in particular reverse transcriptase) inhibiting amount of the derivative(s) of formula I as defined herein corresponds to an amount which ensures a plasma level of between 1 μg/ml and 100 mg/ml, optionally of 10 mg/ml. Depending upon the pathologic condition to be treated and the patient's condition, the said effective amount may be divided into several sub-units per day or may be administered at more than one day intervals.

The present invention further relates to a method for preventing or treating a viral infection, e.g. a retroviral infection, in a subject or patient by administering to the patient in need thereof a therapeutically effective amount, e.g. an anti-virally effective amount, of a compounds of the present invention. The therapeutically effective amount of the compound(s), especially for the treatment of viral infections in humans and other mammals, preferably is a HIV enzyme inhibiting amount. More preferably, it is a HIV replication inhibiting amount or a HIV enzyme (in particular reverse transcriptase) inhibiting amount of the derivative(s) of represented by the structural formula (I) or the structural formula (A) as defined herein. Depending upon the pathologic condition to be treated and the patient's condition, the said effective amount may be divided into several sub-units per day or may be administered at more than one-day intervals.

The present invention also relates to a combination of different antiviral drugs of the invention or to a combination of the antiviral drugs of the invention with other drugs that exhibit anti-HIV.

The invention also relates to a combined preparation of antiviral drugs which may be either:
A) a composition comprising
   (a) a combination of two or more of the compounds of the present invention, including any one of the specific embodiments thereof, and
   (b) optionally one or more pharmaceutical excipients or pharmaceutically acceptable carriers,
      for simultaneous, separate or sequential use in the treatment or prevention of a viral infection, e.g. a retroviral infection, or
B) a composition comprising
   (c) one or more anti-viral agents, and
   (d) at least one compound of the present invention, including any one of the specific embodiments thereof, and
   (e) optionally one or more pharmaceutical excipients or pharmaceutically acceptable carriers,
      for simultaneous, separate or sequential use in the treatment or prevention of a viral infection, e.g. a retroviral infection.

Suitable anti-viral agents (c) for inclusion into the antiviral combined preparations of this invention include for instance, inhibitors of BVDV or HCV replication respectively, such as interferon-alpha (pegylated or not), ribavirin and other selective inhibitors of the replication of HCV, such as a compound disclosed in EP-1,162,196, WO 03/010141, WO 03/007945, WO 03/010140 or WO 00/204425 (the contents of which are incorporated herein by reference) and/or an inhibitor of flaviviral protease and/or one or more additional flavivirus polymerase inhibitors.

The pharmaceutical composition or combined preparation with activity against viral infection according to this invention may contain a compound of the present invention, including any one of the specific embodiments thereof, over a broad content range depending on the contemplated use and the expected effect of the preparation. Generally, the content of the compound of the present invention including any one of the specific embodiments thereof, in the combined preparation is within the range of 0.1 to 99.9% by weight, preferably from 1 to 99% by weight, more preferably from 5 to 95% by weight.

When using a pharmaceutical composition of combined preparation:

the active ingredients may be administered to the mammal (including a human) to be treated by any means well known in the art, i.e. orally, intranasally, subcutaneously, intramuscularly, intradermally, intravenously, intra-arterially, parenterally or by catheterization; and/or

the therapeutically effective amount of each of the active agents, especially for the treatment of viral infections in humans and other mammals, particularly is a HIV enzyme inhibiting amount.

When applying a combined preparation, the active ingredients may be administered simultaneously but it is also beneficial to administer them separately or sequentially, for instance within a relatively short period of time (e.g. within about 24 hours) in order to achieve their functional fusion in the body to be treated.

The invention also relates to the compounds of the formulae described herein, including any one of the above-described specific embodiments thereof, for use in the inhibition of the proliferation of other viruses than HIV-1, particularly for the inhibition of other members of the family of the retroviruses.

The present invention further provides veterinary compositions comprising at least one active ingredient as above defined together with a veterinary carrier therefor. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

More generally, the invention relates to the compounds represented by the structural formula (I) or the structural formula (A), including any one of the above-described specific embodiments thereof, being useful as agents having biological activity (particularly antiviral activity) or as diagnostic agents.

The compounds of the present invention may for instance be bound covalently to an insoluble matrix and used for affinity chromatography separations, depending on the nature of the groups of the compounds, for example compounds with pendant aryl are useful in hydrophobic affinity separations.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds represented by the structural formula (I) or the structural formula (A), including any one of the above-described specific embodiments thereof, are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds represented by the structural formula (I) or the structural formula (A) as well as their salts are able to form, such as for example hydrates, alcoholates and the like. Finally, it is to be understood that the compositions herein comprise compounds of the invention in their unon-ionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NA₄⁺ (wherein A is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NA₄⁺ (wherein A typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

With respect to stereoisomers, more particularly, stereogenic centers may have either the R- or S-configuration, and multiple bonds may have either cis- or trans-configuration. Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question. Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester or Mosher ester, a-methoxy-a-(trifluoromethyl)phenyl acetate (Jacob III. (1982) J. Org. Chem. 47:4165) of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (e.g. WO96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like.

The terms cis and trans are used herein in accordance with Chemical Abstracts nomenclature and include reference to the position of the substituents on a ring moiety. The absolute stereochemical configuration of the compounds of formula I may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulfonates and sulfates; sulfonated benzimidazole derivatives and alkylarylsulfonates. Fatty sulfonates or sulfates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulfonic acid or dodecylsulfonic acid or a mixture of fatty alcohol sulfates obtained from natural fatty acids, alkaline or alkaline-earth metal salts of sulfuric or sulfonic acid esters (such as sodium lauryl sulfate) and sulfonic acids of fatty alcohol/ethylene oxide adducts. Suitable sulfonated benzimidazole derivatives preferably contain 8 to 22 carbon atoms. Examples of alkylarylsulfonates are the sodium, calcium or alcanolamine salts of dodecylbenzene sulfonic acid or dibutyl-naphthalenesulfonic acid or a naphthalene-sulfonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphosphatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulfonates and dialkyl-sulfosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with polypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypoly-ethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C₈₋₂₂ alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing, Ridgewood, New Jersey, 1981), ‘Tensid-Taschenbucw’, 2^{nd} ed. (Hanser Verlag, Vienna, 1981) and “Encyclopaedia of Surfactants” (Chemical Publishing Co., New York, 1981).

Compounds of the invention and their physiologically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

This invention includes controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

This invention shows that a modified nucleoside such as, but not limited to; 2′-deoxy-adenosine-5′-iminodiacetate-phosphoramidate (IA-dAMP) is successfully recognized and efficiently incorporated into a growing DNA strand by HIV RT. This means that an iminodiacetate-phosphoramidate moiety can mimic a pyrophosphate group and behave as a good leaving group in a nucleotidyl transfer. Incorporation of phosphor-amidate or diester analogues, although to a lesser extent, was also observed for phtalic acid phosphoramidates and phosphodiesters, respectively. Therefore, it is feasible to use chain-terminating nucleotides coupled to the novel leaving groups of this invention through a phosphoramidate or phosphodiester linkage for a direct inhibition of HIV RT or other viral polymerases as depicted in FIG. 8A. Effective inhibition of HIV RT or other viral polymerases by a modified nucleoside requires its activation by cellular nucleoside kinases and conversion into a corresponding nucleoside triphosphate. Administration of an AZT analogue as a substitute for AZT nucleoside triphosphate can therefore eliminate a requirement for kinase activation. However, it is important to assess the ability of HIV RT to recognize and insert said AZT analogue with satisfactory efficiency. A potential drawback of this approach could be the charged nature of these nucleosides. As a charged molecule, these AZT analogues are not likely to pass through a cellular membrane unless active transport is involved. However, intracellular diffusion is likely facilitated by masking the negative charges of carboxylate moieties by means of esterification as shown in FIG. 10 (bottom). Once a protected AZT analogue is in the cytosol, it can be transformed back to a charged, acidic form through the action of cellular esterases. These principles of monophosphate activation and subsequent inhibition of viral polymerases, as shown in FIG. 10 for AZT, equally apply to other chain-terminating nucleotides known in the art.

The propagation of new information systems in vivo for synthetic biology purposes, requires that the system is orthogonal to the existing natural informational systems (DNA and RNA). Only in this case, it can be avoided that the natural system will become infiltrated by information from outside. This also applies to the precursors for the enzymatic synthesis of artificial nucleic acids, which means that modified building blocks used for the in vivo synthesis of artificial nucleic acids should not enter the cellular metabolic pathways, since it may lead to toxicity. One way to realize this is to develop an independent metabolic route for chemically modified polymerase substrates. This might be based on the selection of new leaving groups for the polymerase catalyzed nucleic acid synthesis. Preferentially, this leaving group is metabolically available and can be recycled after the reaction.

A better leaving group capacity of an oxygen linked moiety may compensate for a loss in electrostatic effects. This is confirmed when using leaving groups with an aromatic character. In this context, the difference between the phthalic acid and isophthalic acid embodiments described below may be related to a different way that these molecules can be accommodated in the active site of the polymerase.

Using iminodiacetate dAMP as phosphoramidate (IA-dAMP), better incorporation and elongation results are obtained, probably due to the additional flexibility and to a better Mg²⁺ complexation capacity of this leaving group. The incorporation capacity of the analogues seems to be correlated with the value of dissociation constants of the complexes formed by Mg²⁺ and the corresponding dicarboxylic acid.

### Manufacture of the Compounds of the Invention

The synthesis of the modified nucleoside analogues of this invention may be accomplished according to the method illustrated by scheme 2 below, starting from a nucleoside monophosphate, which itself can be tailor-made by phosphorylation of a suitable nucleoside.

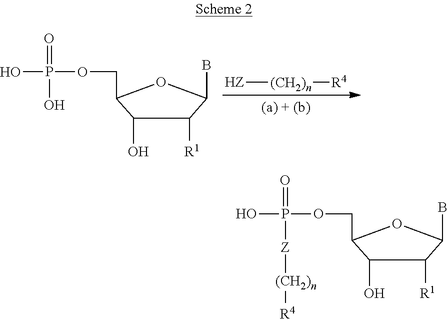

The compounds according to the invention may be synthesized by derivatisation of the 5′-mono-phosphate nucleoside precursor molecule as illustrated in Scheme 2. In a first step (a), the phosphate group of the 5′-mono-phosphate nucleoside is coupled with the Z-group of a reagent represented by the structural formula (E) for producing compounds according to the structural formula (II):

HZ—(CH₂)ₙ—R⁴ (E)

wherein Z is NR^{S}, and wherein n, m, R⁴, R⁵ and R¹¹ are the same as defined hereinabove with respect to formula (II), including any one of the above-described more specific embodiments thereof,
or a reagent represented by the structural formula (F) for producing compounds according to the structural formula (V):

HZ—(CH₂)ₙ—Ar (F)

wherein Z is selected from the group consisting of O, S, NH and NCH₃ and wherein Ar is an aryl group as defined hereinabove with respect to formula (V), including any one of the above-described more specific embodiments thereof.

Specifically, with regard to formula (F), the reagent may be:
an aromatic alcohol (n=0, Z═O) such as, but not limited to, phenol, naphthol and substituted versions thereof;
a benzyl alcohol (n=1, Z═O) being optionally substituted on the phenyl ring;
a 2-phenylethanol (n=2, Z═O) being optionally substituted on the phenyl ring;
an optionally substituted aniline (n=0, Z═NH) being optionally substituted on the phenyl ring such as, but not limited to, an alkoxy-aniline or a halogenated aniline;
a benzylamine (n=1, Z═NH) being optionally substituted on the phenyl ring;
a N-methyl benzylamine (n=1, Z═NCH₃) being optionally substituted on the phenyl ring, such as but not limited to o-methoxy N-methyl benzylamine or p-methoxy N-methyl benzylamine; or
2-phenylethylamine (n=n2, Z═NH).

Representative examples of alkoxy-anilines suitable as starting materials for the first step reaction of the method of scheme 2 include, but are not limited to, 2-methoxyaniline, 3-methoxyaniline, 4-methoxyaniline, 2-ethoxyaniline, 3-ethoxyaniline, 4-ethoxyaniline, 4-bromo-3-ethoxyaniline hydrochloride, 2-propoxyaniline, 3-propoxy-aniline, 4-propoxyaniline, 3-isopropoxyaniline, 4-isopropoxyaniline, 2,5-diethoxyaniline, 3,4-diethoxyaniline, 4-n-butoxyaniline, 3-n-butoxyaniline, 2-n-butoxyaniline, 4-iso-butoxyaniline, 3-isobutoxyaniline, 2-isobutoxyaniline, 2-methyl-4-methoxyaniline, 2-(methylthio)aniline, 3-(methylthio)aniline, 4-(methylthio)aniline, 2-trifluoromethoxyaniline, 3-trifluoromethoxyaniline, 4-trifluoromethoxyaniline, 5-chloro-2-(methylthio)aniline, 2-bromo-4-methoxyaniline, 2-bromo-5-methoxyaniline, 3-bromo-4-methoxyaniline, 4-bromo-3-methoxyaniline, 5-bromo-2-methoxyaniline, 2-iodo-5-methoxyaniline, 3-iodo-4-methoxyaniline, 5-iodo-2-methoxyaniline, 2-chloro-5-methoxyaniline, 3-chloro-2-methoxyaniline, 3-chloro-4-methoxyaniline, 4-chloro-3-methoxyaniline, 5-chloro-2-methoxyaniline, 2-fluoro-4-methoxyaniline, 2-fluoro-6-methoxyaniline, 3-fluoro-2-methoxyaniline, 3-fluoro-4-methoxyaniline, 3-fluoro-5-methoxyaniline, 4-fluoro-3-methoxyaniline, 5-fluoro-2-methoxyaniline, 2-(difluoromethoxy)aniline, 3-(difluoromethoxy)aniline, 4-(difluoromethoxy)aniline and 2,4-dichloro-5-methoxyaniline.

Halo-substituted anilines suitable as starting materials for the first step reaction of the method of scheme 2 include, but are not limited to, 2-fluoroaniline, 3-fluoroaniline, 4-fluoroaniline, 2,3-difluoroaniline, 2,4-difluoroaniline, 2,5-difluoroaniline, 2,6-difluoroaniline, 3,4-difluoroaniline, 2,3,4-trifluoroaniline, 2,3,5-trifluoroaniline, 2,3,6-trifluoroaniline, 2,4,6-trifluoroaniline, 2,4,5-trifluoroaniline, 3,4,5-trifluoroaniline, 3-chloro-2-fluoroaniline, 4-chloro-2-fluoroaniline, 5-chloro-2-fluoroaniline, 2-chloro-3-fluoroaniline, 2-chloro-4-fluoroaniline, 2-chloro-6-fluoroaniline, 3-chloro-5-fluoroaniline, 2-bromo-3-fluoroaniline, 2-bromo-4-fluoroaniline, 2-bromo-5-fluoroaniline, 2-bromo-6-fluoroaniline, 4-bromo-2-fluoroaniline, 4-bromo-3-fluoroaniline, 5-bromo-2-fluoroaniline.

Alkyl-substituted anilines suitable as starting materials for the first step reaction of the method of scheme 2 include, but are not limited to, 4-methylaniline, 3-methylaniline, 2-methylaniline, 2,3-dimethylaniline, 2,4-dimethylaniline, 2,5-dimethylaniline, 2,6-dimethylaniline, 3,4-dimethylaniline, 3,5-dimethylaniline, 2,4,6-trimethylaniline, 3,4,5-trimethylaniline, 2,4,5-trimethylaniline, 2,4,6-triethylaniline, 4-ethylaniline, 3-ethylaniline, 2-ethylaniline, 2-n-propylaniline, 4-n-propylaniline, 2-isopropylaniline, 3-isopropylaniline, 4-isopropylaniline, 2,6-diisopropylaniline, 2-n-butylaniline, 4-n-butylaniline, 2-sec-butylaniline, 4-sec-butylaniline, 2-tert-butylaniline, 3-tert-butylaniline, 4-tert-butylaniline, 3,5-di-tert-butylaniline, 4-n-pentylaniline, 4-n-hexylaniline, 4-n-heptylaniline, 4-n-octylaniline, 4-nonylaniline, 4-n-decylaniline and 4-n-dodecylaniline.

Other substituted anilines suitable as starting materials for the first step reaction of the method of scheme 2 include, but are not limited to, 4-cyclohexylaniline; 2-nitroaniline, 3-nitroaniline, 4-nitroaniline, 2-trifluoromethylaniline, 3-trifluoromethylaniline, 4-trifluoromethylaniline, and the like.

Benzyl alcohols suitable as starting materials for the first step reaction of the method of scheme 2 include, but are not limited to, 2-nitrobenzyl alcohol, 3-nitrobenzyl alcohol, 4-nitrobenzyl alcohol, 4-methylbenzyl alcohol, 4-isopropylbenzyl alcohol, 4-tert-butylbenzyl alcohol, 4-methoxybenzyl alcohol, 3-methoxybenzyl alcohol, 2-methoxybenzyl alcohol, 3-isopropoxybenzyl alcohol, 4-n-butoxybenzyl alcohol, 2-bromobenzyl alcohol, 3-bromobenzyl alcohol, 4-bromobenzyl alcohol, 3,5-dibromobenzyl alcohol, 3-chlorobenzyl alcohol, 2-chlorobenzyl alcohol, 3,4-dichlorobenzyl alcohol, 3,5-dichlorobenzyl alcohol, 2,4-dichlorobenzyl alcohol, 2,6-dichlorobenzyl alcohol, 2,3-dichlorobenzyl alcohol, 4-fluorobenzyl alcohol, 3-fluorobenzyl alcohol, 2-fluorobenzyl alcohol, 2,3-difluorobenzyl alcohol, 3,4-difluorobenzyl alcohol, 3,5-difluorobenzyl alcohol, 2,4-difluorobenzyl alcohol, 2,5-difluorobenzyl alcohol, and 2,6-difluorobenzyl alcohol.

Suitable commercial benzylamines for use in the first step of scheme 2 include, but are not limited to, 2-chlorobenzylamine, 4-chlorobenzylamine, 2,4-dichlorobenzylamine, 3,4-dichlorobenzylamine, 4-methoxybenzylamine, 4-methylbenzylamine, piperonylamine, 3,4-dimethoxybenzylamine, 3-methylbenzylamine, 3-fluorobenzylamine, 2-methylbenzylamine, 2-methoxybenzylamine, 3-methoxybenzylamine, 2-fluorobenzylamine, 4-fluorobenzylamine, 3,4-dihydroxybenzylamine, 3-chlorobenzylamine, 4-(trifluoromethoxy)benzylamine, 2,6-difluorobenzylamine, 3,5-bis(trifluoromethyl)benzylamine, 2,4-difluorobenzylamine, 2,5-difluoro benzylamine, 3,4-difluorobenzylamine, 2-(trifluoromethyl)benzylamine, 3-(trifluoromethyl)benzylamine, 2-bromobenzylamine, 4-bromobenzylamine, 2-chloro-6-fluorobenzylamine, 2,5-dimethylbenzylamine, 3,4,5-trimethoxybenzylamine, 2,4,6-trimethylbenzylamine, 2,4-dimethylbenzylamine, 2,3-dichlorobenzylamine, 1-naphthalenemethylamine, 3-Iodobenzylamine, 2-hydroxybenzylamine, 3-bromo benzylamine, 2,6-dichlorobenzylamine, 3,4-dihydro-2H-1,5-benzodioxepin-6-ylmethylamine, 2,3-dihydro-1,4-benzodioxin-6-ylmethylamine, 2,3-dihydro-1,4-benzodioxin-5-ylmethylamine, 1-benzofuran-5-ylmethylamine, 4-(2-thienyl)benzylamine, 3,4-dihydro-2H-1,5-benzodioxepin-7-ylmethylamine, 4-morpholino benzylamine, 4-(1H-pyrazol-1-yl)benzylamine, 4-(4-methylpiperazino)benzylamine, 2-piperidinobenzylamine, 3-(1H-Pyrrol-1-yl)benzylamine, 2-Morpholinobenzylamine, 4-(1H-pyrrol-1-yl)benzylamine, 2-chloro-6-phenoxy benzylamine, 2-(methylthio)benzylamine, 2-(trifluoromethoxy)benzylamine, 2,3-dimethylbenzylamine, 4-(trifluoromethyl)benzylamine, 3,5-dichlorobenzylamine, 2-(Aminomethyl)-3-fluoroaniline, 3-chloro-4-fluorobenzylamine, 2,5-dimethoxybenzylamine, 2,5-dichloro benzylamine, 2,6-dimethoxybenzylamine, 2,4-dichloro-6-methylbenzylamine, 3-chloro-4-methylbenzylamine, 4-fluoro-3-(trifluoromethyl)benzylamine, 4-fluoro-2-(trifluoromethyl)benzylamine, 3-piperidin-1-ylmethyl benzylamine, 1-benzothiophen-5-ylmethylamine, 4-(Morpholinomethyl)benzylamine, (3-((4-methylpiperidino)methyl)phenyl)methanamine, (4-Piperidinophenyl)methylamine, (3-piperidinophenyl)methylamine, 1-[2-(4-methylpiperazin-1-yl)phenyl]methanamine, (1,4-dimethyl-1,2,3,4-tetrahydroquinoxalin-6-yl)methylamine, 3-(Trifluoromethoxy)benzylamine, 4-bromo-2-fluorobenzylamine, 2-(1 h-pyrazol-1-yl)benzylamine, tert-butyl 4-(2-(aminomethyl)phenyl)piperazine-1-carboxylate, (3-Morpholinophenyl)methylamine, tert-Butyl N-[4-(aminomethyl)phenyl]carbamate, [2-(1H-Pyrrol-1-yl)phenyl]nethylamine, 1-[3-(4-Methylpiperazin-1-yl)phenyl]nethanamine, [4-(1-pyrrolidinyl)phenyl]methanamine, (3-pyrrolidin-1-ylphenyl)methylamine, [4-(2-morpholinoethoxy)phenyl]methylamine, [2-(2-Morpholinoethoxy)phenyl]methylamine, [3-(2-Morpholinoethoxy)phenyl]methylamine, [3-(morpholinomethyl)phenyl]methylamine, [4-(piperidinomethyl)phenyl]methylamine, {4-[(4-Methylpiperazin-1-yl)methyl]phenyl}methylamine, [4-(2-furyl)phenyl]methylamine, tert-Butyl 4-[4-(aminomethyl)phenyl]tetrahydro-1(2H)-pyrazinecarboxylate, (2,2-dimethyl-2,3-dihydro-1-benzofuran-7-yl)methylamine, [3-(1h-1,2,4-triazol-1-yl)phenyl]nethylamine, (4-thien-3-ylphenyl)methylamine, 1-[2-(morpholin-4-ylmethyl)phenyl]methanamine, {2-[(4-methylpiperazin-1-yl)methyl]phenyl}methylamine, [3-(2-furyl)phenyl]methylamine, (3-thien-2-ylphenyl)methylamine, [2-(2-furyl)phenyl]methylamine, 4-(Pyrrolidin-1-ylmethyl)benzylamine, 4-[(4-methylperhydro-1,4-diazepin-1-yl)methyl]benzylamine, 4-[2-(dimethylamino)ethoxy]benzylamine, (2-Pyrrolidin-1-ylphenyl)methylamine, [3-(1-Pyrrolidinylmethyl)phenyl]nethanamine, (3-thien-3-ylphenyl)methylamine, 2-[2-(dimethylamino)ethoxy]benzylamine, 2-(phenoxymethyl)benzylamine, (1-methyl-1h-indol-4-yl)methylamine, 4-(4-methylperhydro-1,4-diazepin-1-yl)benzylamine, (1-methyl-1H-indol-6-yl)methylamine, [3-(1,3-thiazol-2-yl)phenyl]nethylamine, 3-(1H-pyrazol-1-ylmethyl)benzylamine, (1-methyl-1H-indol-5-yl)methylamine, 3-(phenoxymethyl)benzylamine, 2-morpholino-5-(trifluoromethyl)benzylamine, [4-(1,3-Thiazol-2-yl)phenyl]nethylamine, 3-(1-Methyl-1H-pyrazol-3-yl)benzylamine, 2-(4-Methylperhydro-1,4-diazepin-1-yl)benzylamine, 4-[3-(dimethylamino)propoxy]benzylamine, 3-(2-Methyl-1H-imidazol-1-yl)benzylamine, 4-(2-Methyl-1H-imidazol-1-yl)benzylamine, 2-(2-methyl-1H-imidazol-1-yl)benzylamine, [4-(tetrahydropyran-4-yloxy)phenyl]nethylamine, 3-[3-(dimethylamino)propoxy]benzylamine, 2-[3-(dimethylamino)propoxy]benzylamine, 3-pyrimidin-2-ylbenzylamine, 4-(1-methyl-1H-pyrazol-3-yl)benzylamine, 3-(1-methyl-1h-pyrazol-5-yl)benzylamine and 1-(1-benzothien-7-yl)methanamine.

Specifically, with regard to formula (E), the reagent may be:
a diphenylamine (m=n=0, R⁴ and R¹¹ being both phenyl),
a dibenzylamine (m=n=1, R⁴ and R¹¹ being both phenyl), or
an “imino-acid” or an ester thereof such as iminodiacetic acid, iminodipropionic acid or iminodibutyric acid or their methyl or ethyl esters.

Said coupling reaction results in the formation of a phosphate ester (when Z═O), phosphate amide (when Z═NH, NCH₃ or N—R⁵) or phosphate thioester (when Z═S).

Said coupling reaction may be performed using any coupling agent (also referred to as dehydrating agent) known in the art for esterification or amide formation, in particular using a carbodiimide coupling agent, more in particular using dicyclohexylcarbodiimide (DCC). The coupling reaction is performed at a temperature between room temperature and reflux temperature of the solvent the reaction is performed in. Depending on the nature of R⁴ and R⁵, additional acid, hydroxy or amine functionalities in reagent (E) may be transiently protected to prevent these functionalities from interfering with the condensation reaction between the phosphate acid and Z. Therefore, the synthetic route provides for an optional subsequent step (b) of deprotecting such functionalities. This deprotection step can be carried out with potassium carbonate in methanol-water solution.

Schemes 3 and 4 below illustrate a synthetic route for the synthesis of pyrimidine and purine derived compounds according to the present invention respectively.

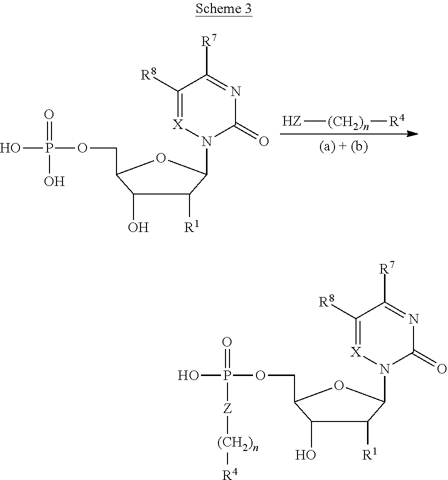

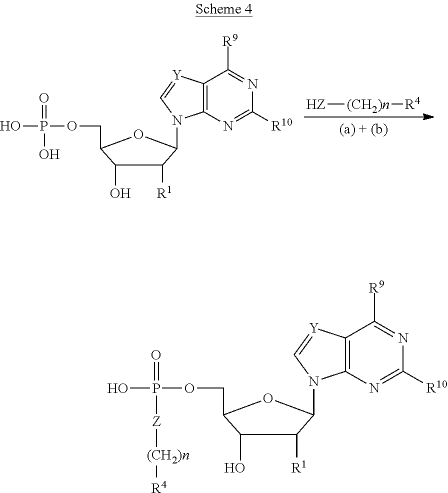

Alternatively, the compounds according to the structural formula I, or any specific embodiments thereof, may be obtained using a synthetic procedure as illustrated by scheme 1 herein above.

The following examples are provided for illustrative purpose only, and should not be considered as limiting the scope of the present invention. The synthesis of the esters of the phosphate-modified nucleosides of the invention was accomplished according to the general principles of the method described by Wagner et al. in Mini-Rev. Med. Chem. (2004) 4:409, starting from a nucleoside monophosphate. Deprotection of the esters was carried out with potassium carbonate in methanol-water solution.

In these examples, the following analytical methods and materials were used. NMR spectral analyses were carried out on a Brucker Avance™ II 300 MHz or 500 MHz with PAXTI probe. The Bruker Topspin™ 2.1 software was used to process spectra. Chemical shifts are expressed in parts per million (ppm) by frequency. 1H and ¹³C NMR chemical shifts are referenced to an internal TMS signal (δ=0.00 ppm), ³¹P NMR chemical shifts are referenced to an external 85% H₃PO₄ standard (δ=0.00 ppm). Standard mass spectra were measured with a Finnigan LCQ DuO (Thermo Fischer Scientific) using the ionisation by electron impact technique (ESI); data were acquired with the LAC/E³² system (Waters). Exact mass spectra were obtained with a Q-T of 2™ (Micromass Ltd.) coupled to a CapLC™ system (Waters). Chemicals of analytical or synthetic grade were obtained from commercial sources and were used as received (deoxyadenosine monophosphate: Sigma Aldrich; dicyclohexylcarbodiimide (DCC), dimethyl iminodiacetic acid hydrochloride: Fluka; tert-butanol and triethylamine: Acros). Technical solvents were obtained from Brenntag (Deerlijk, Belgium). Acetonitrile HPLC Grade was purchased from Fischer Scientific. Flash silica chromatography was performed on Davisil® silica gel 60, 0.040-0.063 mm (Grace Davison). Thin Layer Chromatography was performed on Alugram® silica gel UV254 mesh 60, 0.20 mm (Macherey-Nagel).

Oligodeoxyribonucleotides P1, T1, T2 and T3 were purchased from Sigma Genosys. The concentrations were determined with a Varian Cary-300-Bio UV Spectrophotometer. The lyophilized oligonucleotides were dissolved in diethylpyrocarbonate (DEPC)-treated water and stored at −20° C. The primer oligonucleotides were 5′-³³P-labeled with 5′-[γ³³P]-ATP (Perkin Elmer) using T4 polynucleotide kinase (New England Biolabs) according to standard procedures. The labeled oligonucleotide was further purified using Illustra™ Microspin™ G-25 columns (GE Healthcare).

DNA polymerase reactions: end-labeled primer was annealed to its template by combining primer and template in a molar ratio of 1:2 and heating the mixture to 70° C. for 10 minutes followed by slow cooling to room temperature over a period of 1.5 hour. For the incorporation of a modified nucleoside, a series of 20 μL-batch reactions was performed with the enzyme HIV-1 RT (Ambion, 10 U/μL stock soln, specific activity 8.095 U/mg, concentration 1.2 mg/mL). The final mixture contained 125 nM primer template complex, RT buffer (250 mM Tris.HCl, 250 mM KCl, 50 mM MgCl₂, 2.5 mM spermidine, 50 mM dithiothreitol (DTT); pH 8.3), 0.025 U/μL HIV-1 RT, and different concentrations of phosphoramidate or phosphodiester building blocks (1 mM, 500 μM, 200 μM and 100 μM). In the case of the aromatic analogues 3 and 4, the range of concentrations was limited to 1 mM. In the control reaction with the natural nucleotide, a 10 μM dATP concentration was used. The mixture was incubated at 37° C. and 2.54 aliquots were quenched after 5, 10, 20, 30, 60 and 120 minutes.

The steady-state kinetics of single nucleotide incorporation of the iminodiacetate phosphoramidate 1 (IA-dAMP) and of a natural nucleoside triphosphate (dATP) was determined by gel-based polymerase assay. In all the experiments, the template T1 and the primer P1 were used. The primer and template in 1:2 molar ratio were hybridised in a buffer containing 20 mM Tris.HCl, 10 mM KCl, 2 mM MgSO₄, 0.1% Triton X-100, pH 8.3 and used in an amount to provide 125 nM concentration of the primer in each 20 μL reaction. The range of concentrations for iminodiacetate dAMP was optimized according to a K_{M} value for the incorporation of an individual nucleotide. In the case of HIV-1 RT (Ambion, 10 U/μL), reaction mixtures containing the enzyme in 0.025 U/μL concentration and appropriate substrate concentration to attain 5-25% conversion (nucleoside phosphoramidate or natural dATP) were incubated at 37° C. and run for 8 different time intervals. The reactions were quenched by addition of the buffer 80% formamide, 2 mM EDTA, 1×TBE buffer. The analysis of polymerase reaction was performed by polyacrylamide gel electrophoresis (see detailed protocol below). The incorporation rates (V) were calculated based on the percentage of the extension production (n+1 band). The kinetic parameters (V_{Max} and K_{M} were determined by plotting V (nM/min⁻¹) versus substrate concentration (μM) and fitting the data to a non-linear Michealis-Menten regression using GraphPad Prism Software.

Electrophoresis: all polymerase reaction aliquots (2.5 μL) were quenched by the addition of 10 μL of loading buffer (90% formamide, 0.05% bromophenol blue, 0.05% xylene cyanol and 50 mM ethylenediaminetetraacetic acid (EDTA)). Samples were heated at 85° C. for 5 minutes prior to analysis by electrophoresis for 2.5 hours at 2000 V on a 30 cm×40 cm×0.4 mm 20% (19:1 mono:bis) denaturing gel in the presence of a 100 mM Tris-borate, 2.5 mM EDTA buffer; pH 8.3. Products were visualized by phosphorimaging. The amount of radioactivity in the bands corresponding to the products of enzymatic reactions was determined by using the imaging device Cyclone® and the Optiquant image analysis software (Perkin Elmer).

### Example 1

### Synthesis of a Phosphoramidate Wherein R² is Represented by the Structural Formula (II), R⁵ is Represented by the Structural Formula (III), and R⁴ and R¹¹ are Both Carboxylic Acid Groups

FIG. 1 shows the structure of a representative example of this class (IA-dAMP) and a synthetic scheme including an easy-to-perform two steps reaction to produce it. The synthesis starts from an iminodiacetic acid diester (dimethyl ester shown in FIG. 1, but the skilled person understands that the same reaction may be performed starting from the commercially available diethyl ester or dibenzyl erster as well). It should be noted that the usage of naming amino-acids containing a secondary amine group as “imino acids” is disputed by some, and the IUPAC name for “iminodiacetic acid” is 2-(carboxymethylamino)acetic acid.

### Synthesis of 2′-deoxyadenosine-5′-(dimethyl iminodiacetate) phosphoramidate

In a two neck flask, 2′-deoxyadenosine-5′-monophosphoric acid hydrate (100 mg, 0.286 mmole) and dimethyl iminodiacetate hydrochloride (283 mg, 1.43 mmole) were dissolved in a mixture of 1,4-dioxane (9 ml) and N,N-dimethylformamide (1 ml). A few drops of triethylamine (Et₃N) were added to the solution to facilitate dissolution. Then, a solution of N,N′-dicyclohexylcarbodiimide (DCC) (414 mg, 2.0 mmoles) in 1,4-dioxane (1 ml) was added, the reaction mixture was heated for 3 hours while stirring under nitrogen atmosphere. The progress of the reaction was monitored by TLC (CHCl₃:CH₃OH:H₂O 5:3:0.5). Upon completion, the reaction mixture was cooled down and the solvent was removed by rotary evaporation. The residue was resuspended in water (15 ml), extracted with diethyl ether (3×10 ml), and the aqueous phase was lyophilized. The resulting solid was subjected to column chromatography on silica gel using the following solvent gradient: CHCl₃:CH₃OH (5:2:0, 5:2:0.25, 5:3:0.25, 5:3:0.5). The product was obtained as a white solid (58 mg, 43% yield) and characterized as follows:

¹H NMR (500 MHz, D₂O): δ 8.46 (s, 1H, H₈), 8.24 (s, 1H, H₂), 6.50 (apparent t, 1H, J_{H1′-H2′}=6.5 Hz, H_{1′}), 4.72 (m, 1H, H_{3′}), 4.22 (m, 1H, H_{4′}), 4.00 (m, 2H, H_{5′}), 3.78 (dd, 2H, J=9.5 Hz, CH₂COOCH₃), 3.69 (dd, 2H, J=11 Hz, CH₂COOCH₃), 3.56 (s, 6H, CH₃), 2.89 (m, 1H, H_{2′a}), and 2.61 (m, 1H, H_{2′b}) ppm.

¹³C NMR (125 MHz, D₂O): δ 174.27, 156.07, 153.21, 149.36, 140.38, 119.14, 86.63, 84.12, 72.13, 64.67, 52.62, 49.28, and 39.25 ppm

³¹P NMR (121 MHz, D₂O): δ 6.67 ppm.

HRMS: calculated for C₁₆H₂₂N₆O₉P 473.1186. found: 473.1180.

### Synthesis of 2′-deoxyadenosine-5′-iminodiacetate phosphoramidate (IA-dAMP) (1)

A solution of the 2′-deoxyadenosine-5′-dimethyl iminodiacetate phosphoramidate prepared above (50 mg, 0.105 mmole) in 2 ml of a 0.4 M NaOH solution in MeOH:H₂O, was stirred at room temperature under nitrogen for 2 hours. The progress of the reaction was monitored by TLC (iPrOH/NH₃/H₂O 7:1:2). Upon completion, the reaction mixture was neutralized by addition of 1M TEAA. The solvent was removed under reduced pressure. The residue was purified by column chromatography eluting with the following solvent gradient: CHCl₃:CH₃OH:H₂O 5:2:0, 5:2:0.25, 5:3:0.5, and 5:4:1. The product was isolated and concentrated, affording a white solid (31 mg, 65% yield) which was characterized as follows:

¹H NMR (300 MHz, D₂O): δ 8.46 (s, 1H), 8.12 (s, 1H), 6.09 (d, 1H, J=6.4 Hz, H-1′), 4.77 (m, 1H, H-2′), 4.43 (m, 1H, H-3′), 4.22 (m, 1H, H-4′), 4.01 (m, 1H, H-5′a), 3.85 (m, 1H, H-5′ b), 3.46 (m, 1H, CH₂CH), 2.76 (d, 1H, J=10.8 Hz, Ha-CH₂CH), and 2.35 (d, 1H, J=5.5 Hz, Hb-CH₂CH) ppm,

¹³C NMR (125 MHz, D₂O): δ 171.96, 156.26, 153.35, 149.49, 140.68, 119.41, 86.62, 84.42, 71.87, 64.67, 49.67, and 39.62 ppm.

³¹P NMR (121 MHz, D₂O): δ 8.34 ppm; and

HRMS (ESI): calculated for C₁₄H₁₉N₈O₉P 446.0951. found: 445.0864 (negative mode).

### Example 2

### Synthesis of Phosphoramidates and Phosphates Wherein R² is Represented by the Structural Formula (V) and n=0

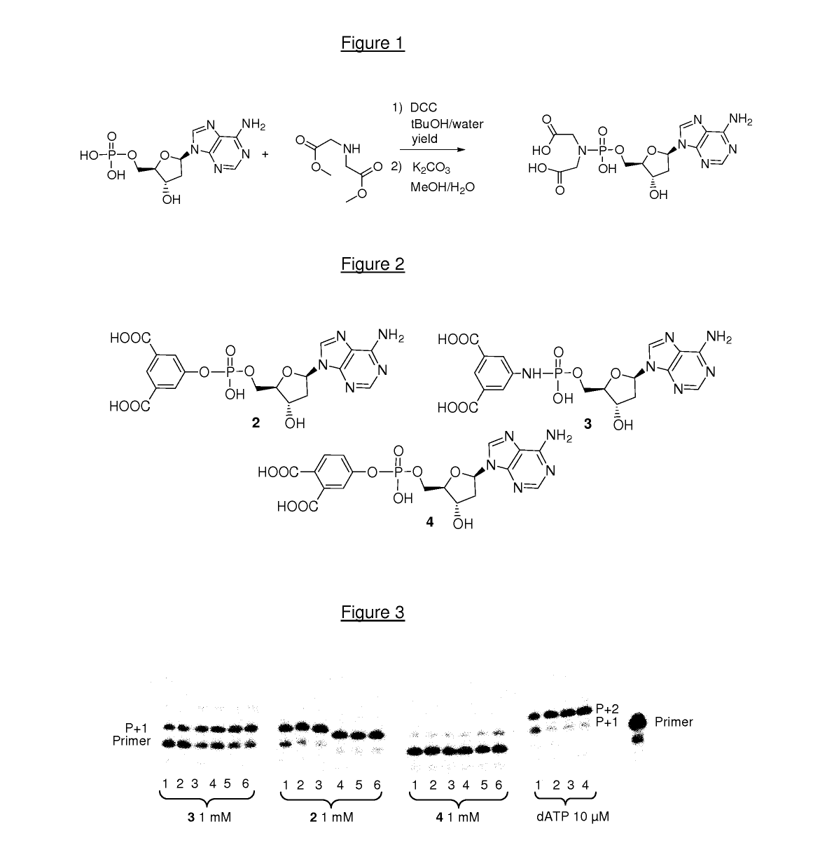

The synthesis of compounds 2-4, the structural representation of which is shown in FIG. 2, was carried out in two steps involving the production of ester intermediates according to the following detailed procedure.

### Synthesis of an ester intermediate, ea 2′-deoxyadenosine-5′-(dimethyl 5-amino-isophthalic acid) phosphoramidate

In a two-neck flask, 2′-deoxyadenosine-5′-monophosphoric acid hydrate (50 mg, 0.1 mmole), 5-amino-isophthalic acid dimethyl ester hydrochloride (245 mg, 1 mmole) and dicyclohexylcarbodiimide (DCC) (147 mg, 0.7 mmol) were dissolved under argon atmosphere in a mixture of tert-butanol (3 mL) and H₂O (1 mL). A few drops of triethylamine (Et₃N) were added to facilitate dissolution. The reaction mixture was refluxed carefully for 6 hours while stirring under argon atmosphere. The progress of the reaction was monitored by thin layer chromatography (TLC) (using a iPrOH:H₂O:NH₃ 7:2:1 eluent mixture). The reaction mixture was cooled down and the solvent was removed by rotary evaporation at 37° C. The resulting product was isolated by silica column chromatography eluting with a CHCl₃:MeOH:H₂O mixture gradient (5:1; 5:2:0.25; 5:3:0.5) affording a white solid (35 mg, 47% yield) which was characterized as follows:

¹H NMR (300 MHz, D₂O): 7.97 (5, 1H, H₈), 7.94 (s, 1H, H_{iPA}), 7.61 (s, 1H, H₂), 7.44 (s, 2H, H_{iPA}), 6.25 (t, J_{H1′-H2′}=6.6 Hz, 1H, H_{1′}), 4.67 (m, 1H, H_{3′}), 4.32 (m, 1H, H_{4′}), 4.08 (m, 2H, H_{5′}), 3.9 (5, 6H, CH₃), 2.85 (m, 1H, H_{2′}), and 2.53 (m, 1H, H_{2″});

¹³C NMR (75 MHz, D₂O): 167.8, 154.6, 151.8, 147.9, 142.4, 139.7, 129.6, 121.7, 86.1, 83.9, 71.1, 65.2, 58.8, 52.7, 46.6, and 38.2;

³¹P NMR (121 MHz, D₂O): 0.22 ppm; and

MS (ESI): [M-H]=521.8, calculated for C₂₀H₂₃N₆O₉P=522.4.

### Synthesis of 2′-deoxyadenosine-5′-(5-aminoisophthalic acid) phosphoramidate (3) by deprotecting the ester intermediate

A solution (1 mL) of 1.3 M K₂CO₃ (MeOH:H₂O 2:1) was added to 2′-deoxyadenine-5′-(dimethyl 5-aminoisophtalic acid) phosphoramidate (35 mg, 67 nmoles) and the deprotection reaction was carried out at room temperature while stirring under argon atmosphere for 4 hours. The course of the reaction was monitored by TLC (using a iPrOH:H₂O:NH₃ 7:2:1 eluent mixture). Once the starting material has disappeared, the reaction mixture was neutralised by addition of 2M TEAB. The solvent was removed under reduced pressure and the resulting residue was dried by lyophilisation. The resulting product was purified by silica column chromatography eluting with (iPrOH:H₂O:NH₃) gradient, isolated and concentrated by lyophilisation to provide a white solid (20 mg, 60% yield) which was characterized as follows:

¹H NMR (300 MHz, D₂O): 8.08 (s, 1H, H₈), 8.00 (s, 1H, H₂), 7.59 (s, 1H, H_{iPA}), 7.31 (s, 2H, H_{iPA}), 6.29 (t, J_{H1′-H2′}=6.6 Hz, 1H, H_{1′}), 4.55 (m, 1H, H_{3′}), 4.22 (m, 1H, H_{4′}), 4.00 (m, 2H, H_{5′}), 2.80 (m, 1H, H_{2′}), and 2.45 (m, 1H, H_{2″});

¹³C NMR (75 MHz, D₂O): 172.7, 153.7, 150.4, 148.0, 141.4, 140.0, 135.1, 121.0, 119.9, 85.9, 84.0, 71.0, 64.3, and 38.4;

³¹P NMR (121 MHz, D₂O): −0.83 ppm; and

calculated for C₁₈H₁₉N₆O₉P=494.4, MS (ESI) found: [M-H]=493.6.

### 2′-deoxyadenosine-5′-(dimethyl 5-hydroxy-isophthalic acid) phosphodiester

This ester intermediate was obtained (62 mg, 79% yield) by a similar synthetic procedure as above, and was characterized as follows:

¹H NMR (300 MHz, D₂O): 8.23 (s, 1H, H₈), 8.06 (s, 1H, H₂), 8.00 (s, 1H, H_{iPA}) 7.68 (s, 2H, H_{iPA}), 6.40 (t, J_{H1′-H2′}=6.7 Hz, 1H, H₁′), 4.4 (m, 1H, H₃′), 4.27 (m, 1H, H₄′), 4.00 (m, 2H, H₅′), 3.9 (s, 3H, CH₃), 3.8 (s, 3H, CH₃), 2.90 (m, 1H, H₂′), and 2.56 (m, 1H, H₂″);

¹³C NMR (75 MHz, D₂O): 166.9, 151.8, 151.6, 151.6, 139.9, 130.6, 125.6, 125.3, 85.9, 83.9, 71.2, 65.9, 52.9, and 38.2;

³¹P NMR (121 MHz, D₂O): −5.05 ppm; and

MS (ESI): [M-H]=522.3, calculated for C₂₀H₂₂N₅O₁₀P 523.11. found 522.20.

### 2′-deoxyadenosine-5-(5-hydroxy-isophthalic acid) phosphodiester (2)

This compound was obtained (25 mg, 66% yield) from the above ester intermediate, and was characterized as follows:

¹H NMR (300 MHz, D₂O): 8.23 (s, 1H, H8), 8.1 (s, 1H, H2), 7.7 (s, 1H, H_{iPA}), 7.6 (s, 2H, H_{iPA}), 6.46 (t, J_{H1′-H2′}=6.7 Hz, 1H, H₁′), 4.65 (m, 1H, H₃′), 4.32 (m, 1H, H₄′), 4.19 (t, 2H, H₅′), 2.84 (m, 1H, H₂′), and 2.56 (m, 1H, H₂″);

¹³C NMR (75 MHz): 173.7, 155.0, 151.8, 151.7, 149.0, 140.6, 137.5, 125.3, 123.4, 119.0, 96.5, 84.7, 71.8, 66.1, and 38.8;

³¹P NMR (121 MHz, solvent): −4.4 ppm; and

MS (ESI): [M-H]=494.3, calculated for C₁₈H₁₉N₆O₉P 495.08.

### 2′-deoxyadenosine-5′-(dimethyl 4-hydroxy-phthalic acid) phosphodiester

This ester intermediate was obtained (70 mg, 93% yield) by a similar synthetic procedure as above, and was characterized as follows:

¹H NMR (300 MHz, D₂O): 8.12 (s, 1H, H₈), 8.09, (s, 1H, H₂), 7.48 (d, 1H, H_{PA}), 7.16 (d, 1H, H_{PA}), 7.13 (s, 1H, H_{PA}), 6.4 (t, J_{H1′-H2′}=6.8 Hz, 1H, H₁′), 4.70 (m, 1H, H₃′), 4.31 (m, 1H, H₄′), 4.21 (m, 2H, H₅′), 3.9 (s, 3H, CH₃), 3.8 (s, 3H, CH₃), 2.81 (m, 1H, H₂′), and 2.54 (m, 1H, H₂″);

¹³C NMR (75 MHz, D₂O): 169.4, 168.5, 154.3, 154.2, 150.4, 148.2, 140.2, 133.1, 130.8, 124.7, 122.6, 119.9, 118.4, 85.9, 84.0, 71.0, 65.8, 53.3, 53.1, and 38.5;

³¹P NMR (121 MHz, D₂O): −5.42 ppm; and

MS (ESI): [M-H]=522.3.

### 2′-deoxyadenosine-5′-(4-hydroxy-phthalic acid) phosphodiester (4)

This compound was obtained (25 mg, 66% yield) from the above ester intermediate, and was characterized as follows:

¹H NMR (300 MHz, D₂O): 8.32 (s, 1H, H₈), 8.21 (s, 1H, H₂), 7.33 (d, 1H, H_{PA}), 7.12 (s, 1H, H_{PA}), 6.92 (d, 1H, H_{PA}), 6.49 (t, J_{H1′-H2′}=6.7 Hz, 1H, H₁′), 4.68 (m, 1H, H₃′), 4.30 (m, 1H, H₄′), 4.17 (m, 2H, H₅′), 2.88 (m, 1H, H₂′), and 2.61 (m, 1H, H₂″);

¹³C NMR (75 MHz, D₂O): 176.0, 175.2, 155.5, 152.42, 148.7, 139.8, 129.5, 119.5, 118.8, 85.6, 83.9, 71.2, 65.7, and 38.6;

³¹P NMR (121 MHz, D₂O): −4.6 ppm; and

High resolution MS (ESI): m/z calculated for C₁₈H₁₉N₆O₉P 495.08. found 496.83.

### Example 3

### Single Incorporation

HIV-1 Reverse Transcriptase serves, in the HIV-1 viral replication process, as a catalyst and uses deoxynucleotides as substrates. This polymerase is error-prone and thus has a high mutation rate. Here, we evaluated the capacity to incorporate a deoxyadenosine nucleoside into the primer-template complex P1T1 using HIV-1 RT, and some of the above described illustrative compounds of the invention, carrying different leaving groups, as substrates. The initial screening was carried out using a template with an overhang of one thymidine nucleotide followed by three non-pyrimidine bases (Table 1). Incorporation efficiency was analysed by the polyacrylamide gel-based single nucleotide incorporation assay.

TABLE 1
primer-template complexes used in DNA
polymerase reactions.
Single nucleotide incorporation and Kinetic
experiments

| | | |
|---|---|---|
| SEQ ID NO: 1 | P1 | 5′-CAGGAAACAGCTATGAC-3′ |
| SEQ ID NO: 2 | T1 | 3′-GTCCTTTGTCGATACTGTCCCC-5′ |

bold letters indicate the template overhang in the hybridized primer-template duplex.

The isophthalic acid-derived phosphodiester (2) was recognized by HIV-1 RT and efficiently incorporated into a growing primer strand (as shown in FIG. 3) with a conversion to an n+1 strand 90-92% (2) over a period of 2 hours at 1 mM concentration. The corresponding anilino-derived phosphate nucleoside (3) was less well recognized as substrate. Finally, little incorporation (13% n+1 product after 2 hours) was observed with the phthalic acid dAMP derivative (4).

A few interesting observations can be drawn from this first panel. Despite the geometric constraint brought by the aromatic ring, dicarboxylated phenol and dicarboxylated aniline can still function as leaving groups in a polymerase catalyzed reaction. A phenolate is a better leaving group than the corresponding aniline anion, although it is unclear whether protonation of the nitrogen atom of the anilino group may be involved in the catalytic mechanism.

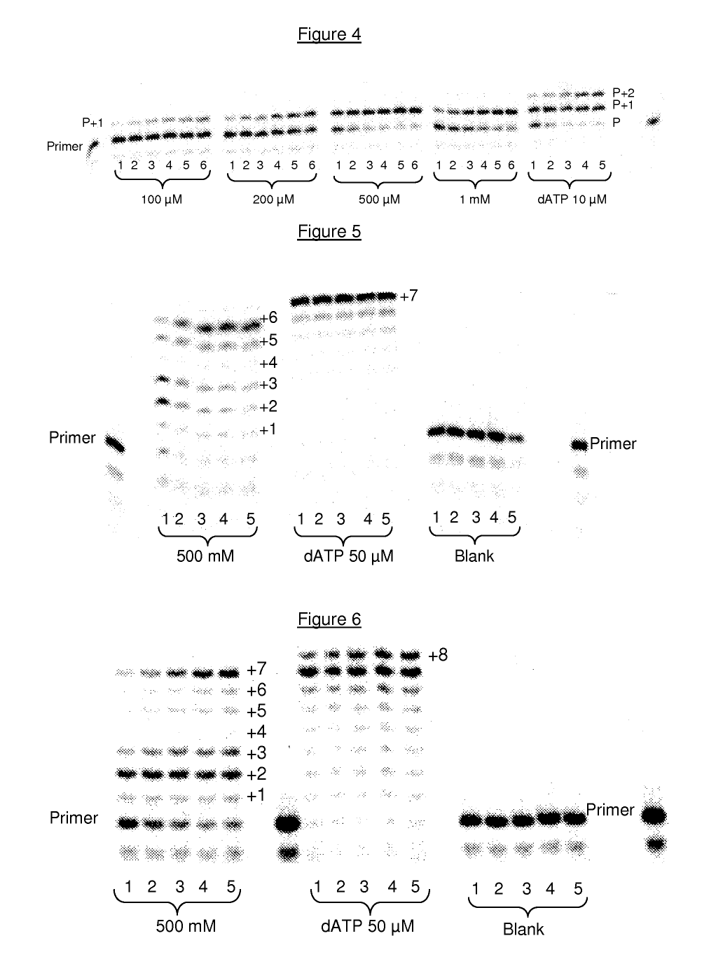

Among the two substituted phenol moieties, the one carrying both carboxyl substituents in meta position (2) is more successful than the one carrying the carboxyl substituents in meta and para positions respectively (4). This indicates that the orientation of both carboxyl functions is important, which might be attributed to steric hindrance in the active site of the polymerase, or to more specific chelating properties. Compound (2) was further evaluated at different concentrations (as shown in FIG. 4). At 500 μM compound (2) displayed 75% of n+1 formation, which represents 88% of L-Asp-dAMP capacity.

We also tested the possibility of a polymerase independent incorporation, but no compound was incorporated in the absence of the enzyme.

### Example 4

### DNA Polymerase Reactions with Compounds (3) and (4)

Oligodeoxyribonucleotides P1, T1, T2 and T3 were purchased from Sigma Genosys. The concentrations were determined with a Varian Cary-300-Bio UV Spectrophotometer.

The lyophilized oligonucleotides were dissolved in diethylpyrocarbonate (DEPC)-treated water and stored at −20° C. The primer oligonucleotides were 5′-³³P-labeled with 5′-[γ³³P]-ATP (Perkin Elmer) using T4 polynucleotide kinase (New England Biolabs) according to standard procedures. The labeled oligonucleotide was further purified using Illustra™ Microspin™ G-25 columns (GE Healthcare).

End-labeled primer was annealed to its template by combining primer and template in a molar ratio of 1:2 and heating the mixture to 70° C. for 10 minutes followed by slow cooling to room temperature over a period of 1.5 hour. For the incorporation of 1, 2, 3, 4, 5 and 6, a series of 20 μL-batch reactions was performed with the enzyme HIV-1 RT (Ambion, 10 U/μL stock solution, specific activity 8.095 U/mg, concentration 1.2 mg/mL). The final mixture contained 125 nM primer template complex, RT buffer (250 mM Tris.HCl, 250 mM KCl, 50 mM MgCl₂, 2.5 mM spermidine, 50 mM dithiothreitol (DTT); pH 8.3), 0.025 U/μL HIV-1 RT, and different concentrations of phosphoramidate or phosphodiester building blocks (1 mM, 500 μM, 200 μM and 100 μM respectively). In the case of the aromatic phosphate-modified nucleosides 3 and 4 of example 2, the range of concentrations was limited to 1 mM. In the control reaction with the natural nucleotide, a 10 μM dATP concentration was used. The mixture was incubated at 37° C. and 2.54 aliquots were quenched after 5, 10, 20, 30, 60 and 120 minutes. Results are shown in FIG. 3.

### Example 5

### Elongation Experiments

For the evaluation of strand elongation capacity of IA-dAMP (compound 1 of example 1), using the same enzyme, the primer-template complexes P1T2 and P1T3 were used as described in Table 2 below.

TABLE 2
overview of the primer-template complexes
used in the elongation experiments.

| | | |
|---|---|---|
| SEQ ID NO: 1 | P1 | 5′-CAGGAAACAGCTATGAC-3′ |
| SEQ ID NO: 3 | T2 | 3′-GTCCTTTGTCGATACTGTTTTTTT-5′ |
| SEQ ID NO: 4 | T3 | 3′-GTCCTTTGTCGATACTGTTTTTTTGGAC-5′ |

Bold letters indicate the template overhang in the hybridized primer-template duplex.

A range of concentrations of the building block were incubated at the appropriate temperature with the primer-template complex and 0.025 U/μL of enzyme. Samples were taken between 5 and 120 minutes and analysed by 20% polyacrylamide gel electrophoresis. In the P1T2 experiment, the template has an overhang of seven thymidine nucleotides. With IA-dAMP (compound 1 of example 1) we observed a prevalence of the (n+6) product, indicating an excellent elongation capacity (as shown in FIG. 5). Initial stalling at the (n+2) and (n+3) products (which disappears in function of time) is much less than when using L-Asp-dAMP as a substrate. We did not observe a complete chain extension to n+7 products when using the P1T2 system.

In the subsequent P1T3 experiment, the seven thymidine bases overhang of the template was prolonged by four non thymidine units. In this case, primer extension up to P+7 was observed (FIG. 6), while misincorporation at the P+8 level was observed when using the natural dATP substrate.

This full elongation, obtained when using the IA-dAMP substrate (compound 1 of example 1), could be due to the increased affinity of the longer template overhang for the enzyme active site and the formation of a more stable ternary complex. It has been shown that the template strand binds to the fingers sub-domain of the polymerase active site. The binding affinity of RT with DNA duplexes is increased when template overhang is extended to 6 additional bases. From models based on crystal structures of the complex, the β3-β4 loop situated in this domain contacts the template three nucleotides away of the primer terminus (near or at Leu⁷⁴). It was postulated that a stronger complex between fingers subdomain of a HIV-RT and the template overhang may be responsible for higher processivity by the enzyme.

Although the binding and incorporation of a natural triphosphate substrate is not dependent on the length of the template overhang, it appears to be the case with our triphosphate analogue. The presence of a four bases overhang at the template's 5′-end could not only favor a more stable dsDNA-protein complex, but in our case, it also influences the ability of HIV-1 RT to recognize and incorporate an incoming dNTP carrying a modified leaving group.

The increased elongation efficiency compared to Asp-dAMP might also be due to the constitutional change i.e. that the leaving nitrogen atom of IA-dAMP is a secondary amine while that of L-Asp-dAMP is a primary amine, and to the better chelating ability of the N-diacetate group of IA-dAMP when compared with the Asp group of L-Asp-dAMP. In the model of Steitz one magnesium ion is coordinated by the 3′ end of the primer and the α-phosphate, while the second magnesium ion is thought to facilitate the leaving group properties of the pyrophosphate moiety by chelating with the β- and γ-phosphates of the nucleotide.

Iminodiacetic acid and aspartic acid display dissociation constants (K_{D 25° C.}) for the divalent magnesium ion of 2.98 and 2.43, respectively, while the dissociation constant of pyrophosphate equals 5.45. A group offering better chelating properties than aspartic acid for the magnesium ion, as is the iminodiacetic acid, might thus act as a better leaving group.

Since in our case, the amino group participates to a phosphoramidate moiety, the contribution of the lone pair on the nitrogen atom to the chelating properties of the leaving group is reduced before the phosphoryl transfer, but not when the P—N bond has been cleaved.

### Example 6

### Kinetic Experiments with Compound 1

Compound 1 (IA-dAMP) was further investigated as follows. Kinetic parameters for the incorporation of both the natural and the modified substrate by HIV-1 RT were determined on the basis of the single completed hit model; P1 and T1 were used as the priming and templating DNA strands, respectively. The kinetic values K_{M} and V_{Max} corresponding to the substrate efficiency of the nucleotides are given in Table 3.

The incorporation efficiency of IA-dAMP for HIV-1 RT is approximately 10⁴ times lower than that of dATP. The K_{M} of IA-dAMP is much higher than for the natural substrate, whereas the V_{Max} is 1.3 folds lower.

TABLE 3
kinetic parameters of the incorporation of dAMP with
the natural pyrophosphate leaving group (dATP) and the iminodiacetate
leaving group (IA-dAMP) into P₁T₁ by HIV-1 RT DNA polymerase
at 0.025 U/μL i.e. 0.047 nM

| | K_{M} | Vₘₐₓ | Vmax/K_{M} |
|---|---|---|---|
| | [μM] | [nM · min⁻¹] | Substrate efficiency |
| dATP | 0.029 ± 0.005 | 65.72 ± 4.30 | 2266*10³ |
| IA-dAMP | 312.5 ± 70.46 | 48.98 ± 4.46 | 0.16*10³ |

### Example 7

### Synthesis of Phosphoramidates Wherein R² is Represented by the Structural Formula (II), R⁵ is Represented by the Structural Formula (III), and R⁴ and R¹¹ are Both Carboxylic Acid Groups

The following two analogues (compounds 5-6, structure shown below) of compound 1 were synthesized for evaluation as potential substrates for HIV-1 RT.

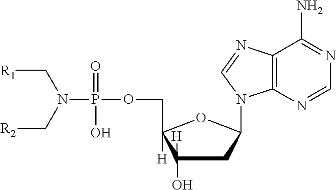

| | R₁ | R₂ |
|---|---|---|
| 5 | CH₂COOH | CH₂COOH |
| 6 | CH₂COOH | COOH |

The synthesis of compounds 5-6 was performed according to the scheme below. Synthesis of methyl ester/ethyl ester phosphoramidate nucleotides analogues were accomplished according to the method described by Wagner and colleagues starting from nucleoside monophosphate

Deprotection of compound 5a and 6a was carried out with sodium hydroxide in methanol-water solution. Scheme 1 showed the synthetic route, which was an easy-to perform two-steps reaction.

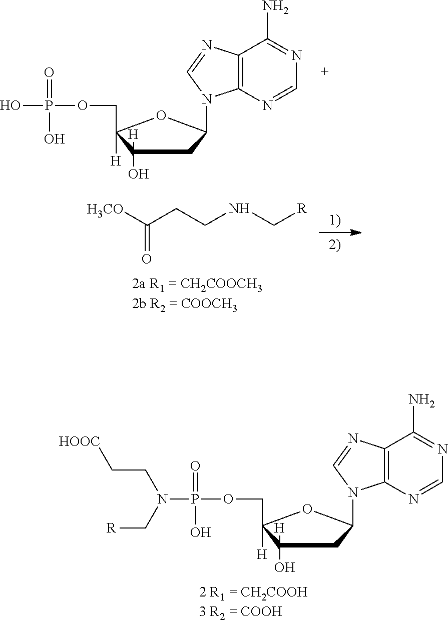

Reaction conditions: step 1) DCC, 1,4-dioxane/DMF at 80° C.; step 2) 0.4M NaOH (MeOH/H₂O).

### Synthesis of 2′-deoxyadenosine-5′-(dimethyl iminodipropionic acid) phosphoramidate (compound 5a)

In a two neck flask, 2′-deoxyadenosine-5′-monophosphoric acid hydrate (100 mg, 0.286 mmoles) and bis(2-carbonyl acid methyl ester) ethyl amine (286 mg, 1.5 mmoles, 5 equiv) were suspended in a mixture of 1,4-dioxane (9 ml) and N,N-dimethylformamide (DMF, 1 ml). A few drops of triethylamine were added to the solution to facilitate dissolution. Then, a solution of DCC (414 mg, 2.004 mmoles, 7 equiv.) in 1,4-dioxane (1 ml) was added and the reaction mixture was heated for 3 hours while stirring under nitrogen atmosphere. The progress of the reaction was monitored by TLC (CHCl₃:CH₃OH:H₂O 5:3:0.5). Upon completion, the reaction mixture was cooled down and the solvent was removed by rotary evaporation. The residue was resuspended in water (15 ml), extracted with diethyl ether (3×10 ml), and the aqueous phase was lyophilised. The resulting solid was subjected to column chromatography on silica gel using the following solvent gradient: CHCl₃:CH₃OH:H₂O (5:1:0; 5:2:0.25; 5:3:0.25; 5:3:0.5). The product was obtained as a white solid (103 mg, 68%) and characterized as follows:

¹H NMR (300 MHz, D₂O): δ 8.50 (s, 1H, H₈), 8.31 (s, 1H, H₂), 6.28 (t, 1H, J=6.4 Hz, H_{1′}), 4.67 (m, 1H, H_{3′}), 4.20 (m, 1H, H_{4′}), 3.95 (m, 2H, H_{5′}), 3.75 (s, 6H, OCH₃), 2.99 (m, 4H, NCH₂CH₂), 2.75 (m, 1H, H_{2′a}), 2.31 (m, 1H, H_{2′b}), and 2.23 (m, 4H, CH2CH₂) ppm;

¹³C NMR (125 MHz, D₂O): 175.1, 155.1, 152.3, 149.8, 139.2, 119.7, 86.4, 83.2, 70.8, 63.5, 52.4, 38.7, 35.2 and 33.1 ppm;

³¹P NMR (121 MHz, D₂O): δ8.28 ppm; and

MS: calculated for C₁₈H₂₈N₈O₉P 501.2. found: 500.9.

### Synthesis of 2′-deoxyadenosine-5′-(iminodipropionic acid) phosphoramidate (compound 5)

A solution of 2′-deoxyadenosine-5′-(dimethyl iminodipropionic acid) phosphoramidate (80 mg, 0.159 mmole) in 0.4 M sodium hydroxide solution in MeOH:H₂O (4:1) (2 mL), was stirred at room temperature under nitrogen for 2 hours. The progress of the reaction was monitored by TLC (iPrOH/NH₃/H₂O 7:1:2). Upon completion, the reaction mixture was neutralised by addition of 1M triethylammonium bicarbonate. The solvent was removed under reduced pressure. The residue was purified by column chromatography and eluted with the following solvent gradient: CHCl₃:CH₃OH:H₂O 5:1:0, 5:2:0.25, 5:3:0.5, and 5:4:1. The product was isolated and concentrated, affording a white solid (64 mg, 85% yield). and characterized as follows:

¹H NMR (300 MHz, D₂O): δ 8.37 (s, 1H, H₈), 8.15 (s, 1H, H₂), 6.41 (t, 1H, J=6.58 Hz, H_{1′}), 4.70 (m, 1H, H_{3′}), 4.18 (m, 1H, H_{4′}), 3.86 (m, 2H, H_{5′}), 3.06 (m, 4H, CH₂CH2COOH), 2.77 (m, 1H, H_{2′a}), 2.53 (m, 1H, H_{2′b}), and 2.27 (m, 4H, CH₂ CH₂COOH) ppm;

¹³C NMR (125 MHz, D₂O): δ 180.73, 155.23, 152.33, 148.39, 139.59, 118.40, 85.76, 83.43, 70.81, 63.54, 43.09, 37.13 and 32.18 ppm;

³¹P NMR (121 MHz, D₂O): δ9.09 ppm; and

HRMS: calculated for C₁₈H₂₂N₈O₉P 473.1191. found: 473.1202.

### Synthesis of 2′-deoxyadenosine-5′-(N,N-carboxymethyl-β-carboxyethyl dimethyl ester) phosphoramidate (compound 6a)

The same general procedure was applied using 2′-deoxyadenosine-5′-monophosphate (100 mg, 0.30 mmole) and bis(2-carbonyl acid methyl ester) ethyl amine (286 mg, 1.5 mmole) and DCC (436 mg, 2.1 mmoles) as the coupling reagent. After purification obtained white solid product (103 mg, 68% yield) and characterized as follows:

¹H NMR (500 MHz, D₂O): δ 8.36 (s, 1H, H₈), 8.15 (s, 1H, H₂), 6.39 (t, 1H, J=6.5 Hz, H_{1′}), 4.65 (m, 1H, H_{3′}), 4.16 (m, 1H, H_{4′}), 3.87 (m, 2H, H_{5′}), 3.60 (dd, 2H, J=11.0 Hz, CH₂COOCH₃), 3.51 (s, 3H, CH₂COOCH₃), 3.45 (s, 3H, CH₂CH₂COOCH₃), 3.07 (m, 2H, CH₂CH₂COOCH₃), 2.79 (m, 1H, H_{2′a}), 2.51 (m, 1H, H_{2′b}) and 2.28 (m, 2H, CH₂CH₂COOCH₃) ppm;

¹³C NMR (125 MHz, D₂O): δ 174.81, 174.19, 155.03, 152.23, 148.48, 139.70, 118.31, 85.81, 83.33, 70.98, 63.78, 51.87, 51.70, 48.83, 43.33, 38.42 and 33.31 ppm;

³¹P NMR (121 MHz, D₂O): δ7.58 ppm; and

HRMS: calculated for C₁₇H₂₄N₈O₉P 487.1348. found: 487.1324.

### Synthesis of 2′-deoxyadenosine-5′-(N,N-carboxymethyl-β-carboxyethyl)phosphoramidate (compound 6)

The same general procedure was applied using 2′-deoxyadenosine-5′-β-alanine (dimethyl iminodipropionic) phosphoramidate (80 mg, 0.16 mmole), 0.4 M sodium hydroxide solution in MeOH:H₂O (4:1) (3 mL), after reaction obtained white solid product (54 mg, 72%) and characterized as follows:

¹H NMR (300 MHz, D₂O): δ 8.38 (s, 1H, H₈), 8.12 (s, 1H, H₂), 6.39 (t, 1H, J=6.57 Hz, H_{1′}), 4.64 (m, 1H, H_{3′}), 4.18 (m, 1H, H_{4′}), 3.92 (m, 2H, H_{5′}), 3.49 (m, 2H, CH₂COOH), 3.15 (m, 2H, CH₂CH₂COOH), 2.77 (m, 1H, H_{2′a}), 2.52 (m, 1H, H_{2′b}) and 2.28 (m, 2H, CH2CH₂COOH) ppm;

¹³C NMR (125 MHz, D₂O): δ 177.79, 171.06, 155.08, 152.18, 148.36, 139.65, 118.38, 85.81, 83.39, 71.02, 63.67, 49.75, 44.56, 38.71, 35.91 and 32.20 ppm;

³¹P NMR (121 MHz, D₂O): δ8.88 ppm; and

HRMS: calculated for C₁₅H₂₀N₆O₉P 459.1035. found: 459.1043

### Example 8

### Biological Evaluation of Phosphoramidates (Compounds 5 and 6)

The ability to incorporate compounds 5-6 into a growing DNA chain was processed by HIV-1 RT. Compound 5 showed the best results, with 90.7% conversion to (P+1) strand after 60 minutes (10-fold lower substrate concentration than L-asp-dAMP) in the single incorporation assay, full elongation was observed in the chain elongation experiment, and kinetics were greatly improved compared to L-asp-dAMP, although the Vmax/K_{M} radio was still 82.8-fold lower than the natural substrate.

The ability of HIV-1 reverse transcriptase to incorporate phosphoramidate analogues 5-6 was analysed by gel-based single-nucleotide-incorporation assays using primer-template complex P₁T₁.

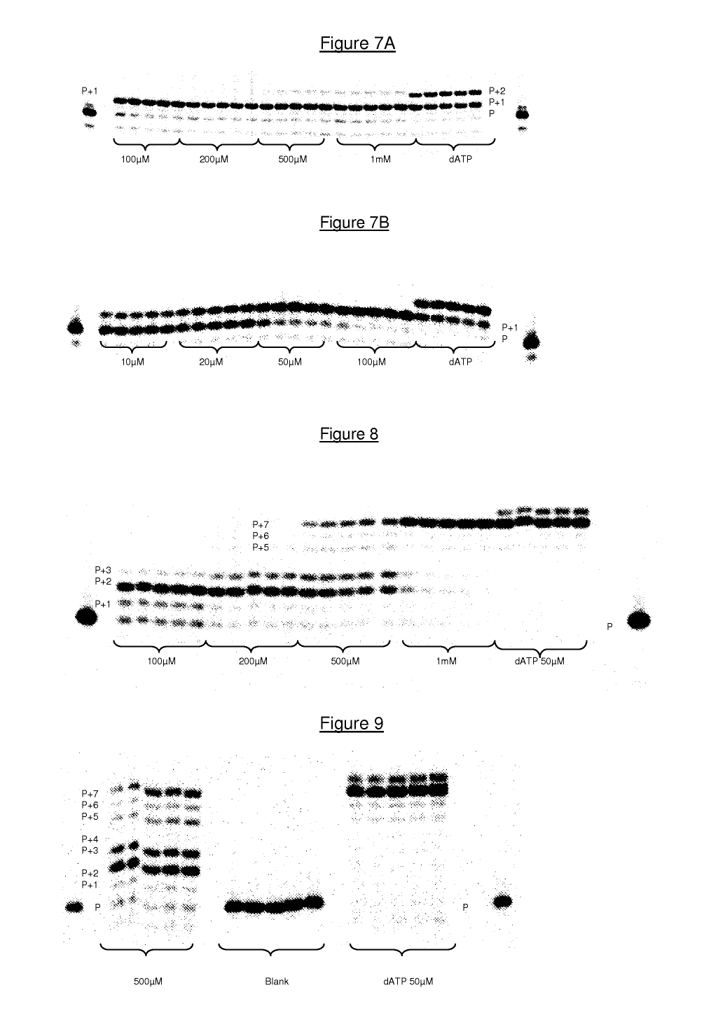

Compound 5 shows the most remarkable result at 50 μM concentration (FIG. 7A). Efficient substrate incorporation can also be detected when substrate concentration decreased to 5 μM or 10 μM, with 17.8% and 33% incorporation, respectively. The corresponding analogue 6 is less well recognized under the same condition, compound 6 can only get 63% incorporation within 60 minutes with a 500 μM substrate concentration (FIG. 7B).

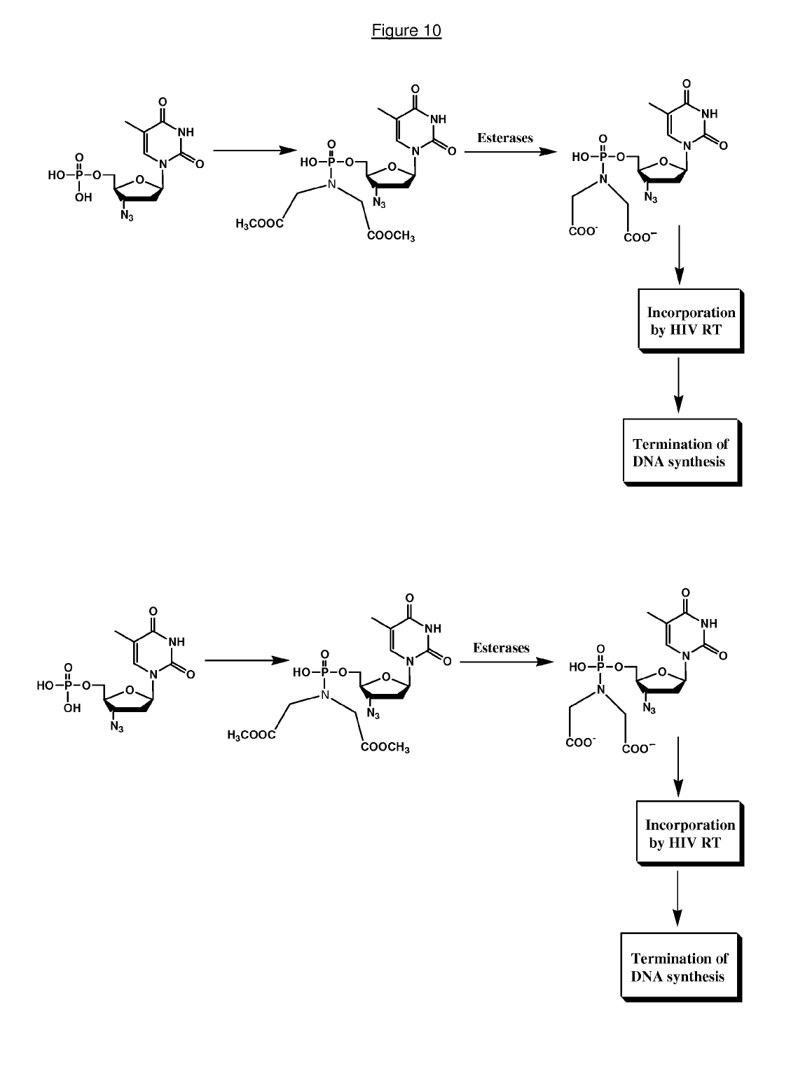

In order to investigate the strand elongation capacity of compound 5, a template dependent incorporation of more than one nucleotide experiment was carried out. In this experiment HIV-1 RT and P₁T₂ duplex, where seven thymidine bases overhang of the template is flanked by four non-thymidine units at the 3′-end were used. A range of concentration of the building block was incubated with the primer-template complex and 0.025 U/μL of enzyme at the appropriate temperature, samples were quenched after 15, 30, 60, 90 and 120 minutes and analysed by 20% polyacrylamide gel electrophoresis. For compound 5 we can get a full-length elongation (FIGS. 8 and 9), with 30% (P+7) product at 500 μM substrate concentration over a period of 2 hours, and 93% (P+7) product at 1 mM substrate concentration without any misincorporation into (P+8) product (FIG. 3), disclosing an excellent elongation capacity. However we can only observe trance amount of (P+7) product at 200 μM substrate concentration, with mainly (P+2) and (P+3) product obtained, and only 8% (P+3) product can observed at 100 μM substrate concentration (FIG. 8).

TABLE 4
compounds 5 and 6 in the elongation of P1 directed by template
T2: % of P + n product after a 120-minutes reaction

| | % product ^{a} | % product ^{a} |
|---|---|---|
| Product | Compound 5 | Compound 6 |
| P + 7 | 28.6 | 0 |
| P + 6 | 5.1 | 0 |
| P + 5 | 7.9 | 0 |
| P + 4 | trances | trances |
| P + 3 | 21.5 | 9.4 |
| P + 2 | 30.2 | 53.7 |
| P + 1 | 3.5 | 9.6 |

^{a} Percentage of the total amount of radio-emitting oligonucleotides in the mixture.

Kinetic parameters of both the natural and the modified substrate by HIV-1 RT were tested on the basis of the single completed hit model. The steady-state kinetic values K_{M} and Vₘₐₓ, corresponding to the substrate efficiency of compound 5 are shown in Table 5. Besides the excellent incorporation and elongation results, compound 5 also showed improved incorporation kinetics than L-Asp-dAMP. As shown in Table 5, the Vₘₐₓ value of compound 5 was similar to the natural substrate, but the K_{M} value was 80-fold higher than dATP, so the ratio of Vmax/K_{M} was 82.8-fold lower than dATP. The Vmax/K_{M} ratio was still lower than dATP but the kinetics of compound 5 still improved a lot compared with L-Asp-dAMP, which showed a Vmax/K_{M} ratio that was 1300-fold lower than the natural substrate.

TABLE 5
Steady-state kinetics: incorporation of compound 5 by HIV-RT

| | Vₘₐₓ | K_{M} | Vmax/K_{M} |
|---|---|---|---|
| | [nM · min⁻¹] | [μM] | [min⁻¹] |
| dATP | 12.37 ± 0.4512 | 0.9346 ± 0.1334 | 13.2356*10⁻³ |
| Compound 5 | 11.78 ± 0.4812 | 73.66 ± 9.869 | 0.1599*10⁻³ |
| (IP-dAMP) | | | |

1-28. (canceled)

29. A phosphate-modified nucleoside represented by the structural formula A: 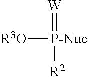
wherein
Nuc is a natural nucleoside or a nucleoside analogue, wherein said natural nucleoside or nucleoside analogue can be non-substituted or substituted;
R³ is selected from the group consisting of hydrogen, C₁₋₆ alkyl, C₃₋₆ cycloalkyl, aryl-C₁₋₆ alkyl, and 2-cyanoethyl; wherein any one of alkyl, cycloalkyl or arylalkyl may optionally be substituted with 1, 2 or 3 substituents independently selected from the group consisting of halogen, OH, C₁₋₆ alkoxy, trifluoromethyl, trifluoromethoxy, nitro, cyano and amino;
W is O or S;
R² is represented by the structural formula (V): 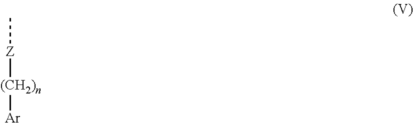
wherein
dotted lines represent the point of attachment of Z to the phosphorous atom P of the structural formula (A);
n is 0, 1 or 2;
Z is selected from the group consisting of O; S; NH and NCH₃; and
Ar is an aryl group,
provided that when W is S and Z is O, n is not 1 or 2,
and stereoisomers, pharmaceutically acceptable salts and pro-drugs thereof,
or R² is represented by the structural formula (II) 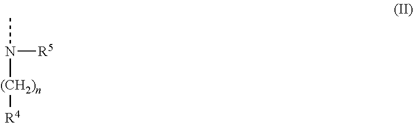
wherein
dotted lines represent the point of attachment of N to the phosphorous atom P of the structural formula (A);
n is 0, 1, 2, or 3;
R⁴ is an aryl group or COOR⁶, wherein R⁶ is hydrogen or C₁₋₆ alkyl or benzyl; and
R⁵ is a group represented by the structural formula (III):
—(CH₂)ₘ—R¹¹ (III)
wherein
the dotted line represents the point of attachment to the nitrogen atom of the structural formula (II);
m is 0, 1, 2, or 3; and
R¹¹ is selected from the group consisting of aryl, imidazolyl and COOR⁶ wherein R⁶ is hydrogen or C₁₋₆ alkyl or benzyl;
or R⁵ is a group represented by the structural formula (IV) 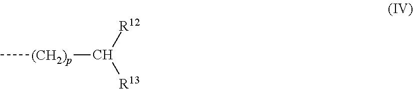
wherein
the dotted line represents the point of attachment to the nitrogen atom of the structural formula (II);
p is 0, 1, 2, or 3; and
each of R¹² and R¹³ is independently selected from the group consisting of
aryl; hydrogen; (CH₂)_{q}-imidazolyl; and (CH₂)_{q}—COOR⁶, wherein R⁶ is hydrogen or C₁₋₆ alkyl or benzyl, and wherein q is 0, 1 or 2, provided that if p is 0, R¹² and R¹³ are not both hydrogen;
and stereoisomers, pharmaceutically acceptable salts and pro-drugs thereof,
provided that said modified nucleoside is not:
2′,3′-didehydro-2′,3′-dideoxyadenosine-5′-phenyl phosphate,
2′,3′-didehydro-2′,3′-dideoxycytidine-5′-phenyl phosphate,
ribofuranosyl-pyrazine carboxamide-5′-dibenzylphosphate,
2′,3′-didehydro-2′,3′-dideoxythymidine-5′-dibenzylphosphate,
2′,3′-didehydro-2′,3′-dideoxythymidine-5′-di(α-methylbenzyl)phosphate,
guanosine 5′-[hydrogen[[4-[(2-chloroethyl)methylamino]phenyl]methyl]-phosphoramidate,
N,N-dimethylguanosine 5′-(S-phenyl hydrogen phosphorothioate),
adenosine 5′-(hydrogen phenylphosphoramidate),
adenosine 5′-[hydrogen (phenylmethyl)phosphoramidate,
uridine 5′-[hydrogen (4-chlorophenyl)phosphoramidate],
uridine 5′-[hydrogen (4-bromophenyl)phosphoramidate], or
uridine 5′-[hydrogen (4-iodophenyl)phosphoramidate.

30. The modified nucleoside of claim 29, being represented by the structural formula (I): 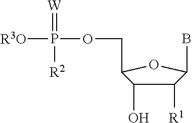
wherein
B is a pyrimidine or purine base, or an analogue thereof, optionally substituted with one or two substituents independently selected from the group consisting of halogen, hydroxyl, sulfhydryl, methyl, ethyl, isopropyl, amino, methylamino, ethylamino, trifluoromethyl and cyano;
R¹ is H or OH;
R³ is selected from the group consisting of hydrogen, C₁₋₆ alkyl, C₃₋₆ cycloalkyl, aryl-C₁₋₆ alkyl and 2-cyanoethyl, wherein said C₁₋₆ alkyl, C₃₋₆ cycloalkyl or aryl-C₁₋₆ alkyl is optionally substituted with one or more, preferably 1, 2 or 3, substituents independently selected from the group consisting of halogen, OH, C₁₋₆ alkoxy, trifluoromethyl, trifluoromethoxy, nitro, cyano and amino;
W is O or S; and
R² is represented by the structural formula (V): 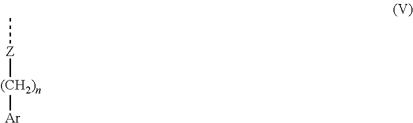
wherein
dotted lines represent the point of attachment of Z to the phosphorous atom P of the structural formula (I);
n is 0, 1 or 2;
Z is selected from the group consisting of O; S; NH and NCH₃; and
Ar is an aryl group,
provided that when W is S and Z is O, n is not 1 or 2, and stereoisomers, pharmaceutically acceptable salts and pro-drugs thereof, or R² is represented by the structural formula (II): 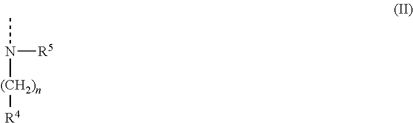
wherein
dotted lines represent the point of attachment of N to the phosphorous atom P of the structural formula (I);
n is 0, 1, 2, or 3;
R⁴ is selected from the group consisting of aryl, imidazolyl and COOR⁶ wherein R⁶ is H or C₁₋₆ alkyl or benzyl;
R⁵ is a group represented by the structural formula (III):
—(CH₂)ₘ—R¹¹ (III)
wherein
the dotted line represents the point of attachment to the nitrogen atom of the structural formula (II);
m is 0, 1, 2, or 3; and
R¹¹ is selected from the group consisting of: aryl, imidazolyl and COOR⁶
wherein R⁶ is hydrogen or C₁₋₆ alkyl or benzyl;
or R⁵ is a group represented by the structural formula (IV): 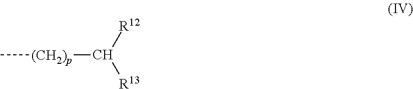
wherein
the dotted line represents the point of attachment to the nitrogen atom of the structural formula (II);
p is 0, 1, 2, or 3; and
each of R¹² and R¹³ is independently selected from the group consisting of aryl; hydrogen; (CH₂)_{q}-imidazolyl; and (CH₂)_{q}—COOR⁶, wherein R⁶ is hydrogen or C₁₋₆ alkyl or benzyl, and wherein q is 0, 1 or 2, provided that if p is 0, R¹² and R¹³ are not both hydrogen, and stereoisomers, pharmaceutically acceptable salts and pro-drugs thereof,
provided that said modified nucleoside is not:
2′,3′-didehydro-2′,3′-dideoxyadenosine-5′-phenyl phosphate,
2′,3′-didehydro-2′,3′-dideoxycytidine-5′-phenyl phosphate,
ribofuranosyl-pyrazine carboxamide-5′-dibenzylphosphate,
2′,3′-didehydro-2′,3′-dideoxythymidine-5′-dibenzylphosphate,
2′,3′-didehydro-2′,3′-dideoxythymidine-5′-di(α-methylbenzyl)phosphate,
guanosine 5′-[hydrogen[[4-[(2-chloroethyl)methylamino]phenyl]methyl]-phosphoramidate,
N,N-dimethylguanosine 5′-(S-phenyl hydrogen phosphorothioate),
adenosine 5′-(hydrogen phenylphosphoramidate),
adenosine 5′-[hydrogen (phenylmethyl)phosphoramidate,
uridine 5′-[hydrogen (4-chlorophenyl)phosphoramidate],
uridine 5′-[hydrogen (4-bromophenyl)phosphoramidate], or
uridine 5′-[hydrogen (4-iodophenyl)phosphoramidate.

31. The modified nucleoside of claim 29 wherein m is 1.

32. The modified nucleoside of claim 29 wherein n is 1.

33. The modified nucleoside of claim 29 wherein R⁴ is COOH and R⁵ is CH₂—COOH.

34. The modified nucleoside of claim 29 wherein R³ is H.

35. The modified nucleoside of claim 29, being selected from the group consisting of 2′-deoxy-adenosine-5′-iminodiacetate-phosphoramidate (IA-dAMP); 2′-deoxy-cytidine-5′-iminodiacetate-phosphoramidate (IA-dCMP); 2′-deoxy-guanosine-5′-iminodiacetate-phosphoramidate (IA-dGMP); 2′-deoxy-thymidine-5′-iminodiacetate-phosphoramidate (IA-dTMP); and 2′-deoxy-uridine-5′-iminodiacetate-phosphoramidate (IA-dUMP).

36. The modified nucleoside of claim 29, being selected from the group consisting of adenosine-5′-iminodiacetate-phosphoramidate (IA-AMP); cytidine-5′-iminodiacetate-phosphoramidate (IA-CMP); guanosine-5′-iminodiacetate-phosphoramidate (IA-GMP); 5-methyluridine-5′-iminodiacetate-phosphoramidate (IA-m5uMP); and uridine-5′-iminodiacetate-phosphoramidate (IA-UMP).

37. The modified nucleoside of claim 29 wherein R³ is H; R⁴ is COOCH₃; and R⁵ is CH₂—COOCH₃.

38. The modified nucleoside of claim 30, wherein said pyrimidine analogue is represented by the structural formula (C): 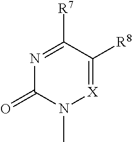
wherein
R⁷ is selected from the group consisting of —OH, —SH, —NH₂, —NHCH₃ and —NHC₂H₅;
R⁸ is selected from the group consisting of hydrogen, methyl, ethyl, isopropyl, amino, ethylamino, trifluoromethyl, cyano and halogen; and
X is CH or N.

39. The modified nucleoside of claim 30, wherein said purine analogue is represented by the structural formula (D): 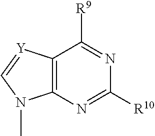
wherein
R⁹ is selected from the group consisting of H, —OH, —SH, —NH₂, and —NHCH₃;
R¹⁰ is selected from the group consisting of hydrogen, methyl, ethyl, hydroxyl, amino and halogen; and
Y is CH or N.

40. The modified nucleoside of claim 29, wherein Nuc is a nucleoside analogue wherein the ribose sugar of said nucleoside is replaced with another monocyclic sugar selected from the group consisting of arabinofuranose, arabinopyranose, xylofuranose, xylopyranose, lyxofuranose, lyxopyranose and α-D-threofuranose.

41. The modified nucleoside of claim 29, wherein Nuc is a nucleoside analogue wherein the deoxyribose sugar of said nucleoside is 2,3-deoxy-3-azido-ribose.

42. The modified nucleoside of claim 40, wherein Nuc is a nucleoside analogue wherein the pyrimidine or purine base of said nucleoside, or an analogue thereof, is optionally substituted with one or two substituents independently selected from the group consisting of halogen, hydroxyl, sulfhydryl, methyl, ethyl, isopropyl, amino, methylamino, ethylamino, trifluoromethyl and cyano.

43. The modified nucleoside of claim 41, wherein Nuc is a nucleoside analogue wherein the pyrimidine or purine base of said nucleoside, or an analogue thereof, is optionally substituted with one or two substituents independently selected from the group consisting of halogen, hydroxyl, sulfhydryl, methyl, ethyl, isopropyl, amino, methylamino, ethylamino, trifluoromethyl and cyano.

44. A substrate for a DNA/RNA polymerase comprising the modified nucleoside of claim 29.

45. The substrate of claim 44 in bacteriae or in vitro.

46. The substrate of claim 44, wherein said polymerase is from a micro-organism or from bacterial or viral origin.

47. The substrate of claim 44, wherein the polymerase is Therminator DNA polymerase; KF (exo⁻) DNA polymerase or Reverse Transcriptase such as HIV-RT.

48. A DNA- or RNA-strand comprising at least one modified nucleoside according to claim 29.

49. A DNA- or RNA-strand according to claim 48, comprising at most 300 of said modified nucleosides.

50. A phosphate-modified nucleoside according to claim 29 for sustaining growth, survival or proliferation of a non-human living organism.

51. A phosphate-modified nucleoside according to claim 50, wherein said organism is selected from the group consisting of a virus, a bacterium, an archaeon and an eukaryote.

52. A phosphate-modified nucleoside according to claim 51, wherein said eukaryote is selected from the group consisting of yeast, mold, fungus, microalga, multicellular plant and protist.

53. A composition comprising a modified nucleoside according to claim 29, an aqueous solution and optionally one or more buffering agents, and optionally one or more nucleoside triphosphates (NTP).

54. The composition of claim 53, wherein said nucleoside triphosphates consists of a mixture of natural NTPs selected from the group consisting of ATP, CTP, GTP, UTP and TTP.

55. A pharmaceutical or veterinary composition comprising an anti-virally effective amount of a modified nucleoside according to claim 29, and one or more pharmaceutically or veterinarilly acceptable excipients.

56. A method of prevention or treatment of a viral infection in a mammal, comprising the administration of an antiviral amount of a modified nucleoside according to claim 29, optionally in combination with one or more pharmaceutically acceptable excipients.

57. The method of claim 56, wherein said viral infection is a HIV infection.

58. An in vitro method of production of DNA, RNA, peptides or proteins, being performed with a wild type or mutated polymerase comprising the modified nucleoside of claim 29.

59. A PCR method performed with a phosphate-modified nucleotides according to claim 29.
